(19) United States
(12) Patent Application Publication
(10) Pub. No.: US 2010/0068260 A1
(43) Pub. Date: Mar. 18, 2010

(54) Methods, Compositions and Device for Directed and Controlled Heating and Release of Agents

(76) Inventors: Dustin E. Kruse, Woodland, CA (US); Claude Meares, Davis, CA (US); Katherine W. Ferrara, Davis, CA (US); Eric Paoli, Los Altos, CA (US); Douglas N. Stephens, Davis, CA (US); Jeffrey Day, (US)

Correspondence Address: FENWICK & WEST LLP, SILICON VALLEY CENTER, 801 CALIFORNIA STREET, MOUNTAIN VIEW, CA, 94041, US

(21) Appl. No.: 12/508,076

(22) Filed: Jul. 23, 2009

Related U.S. Application Data
(63) Continuation-in-part of application No. PCT/US2008/000915, filed on Jan. 23, 2008.
(60) Provisional application No. 60/886,276, filed on Jan. 23, 2007.

(51) Int. Cl. A61K 9/127; A61N 7/02

(52) U.S. Cl. 424/450; 601/3

(57) ABSTRACT

A composition coupled to an agent with a cleavable linker is provided. Specifically, the composition is used for releasing the agent through a temperature-sensitive mechanism at a targeted location in a subject with heat. It is advantageous to applications where there is a need to accurately deploy an agent in a targeted location to reduce adverse side effects or increase efficacy of the agent. A device and method for providing heat at the targeted location in the subject is also provided. The device and method allows release of the agents in a targeted manner and prevents overheating of the targeted location or the tissue surrounding the targeted location. It is advantageous to applications where there is a need to accurately control the temperature in a targeted location in a biological body, for instance, to deploy an agent in the targeted location.

### CROSS REFERENCE TO RELATED APPLICATIONS

This application claims the benefit of U.S. Provisional Application No. 60/886,276, filed Jan. 23, 2007, and PCT application PCT/US2008/00915, filed Jan. 23, 2008, the entire disclosures of which are incorporated by reference in their entirety for all purposes.

### STATEMENT REGARDING FEDERALLY SPONSORED RESEARCH OR DEVELOPMENT

The U.S. Government has certain rights in this invention pursuant to Grant Nos. CA 103828 and CA016861 awarded by the National Institutes of Health.

### BACKGROUND

1. Field of the Invention

The invention relates to the fields of chemistry and biology.

2. Description of the Related Art

Temperature sensitive drug delivery vehicles have been proposed by other labs and have typically used lipid or polymer membranes loaded with drug within the vehicle that leak when heated (Needham, D., N. Stoicheva, et al. (1997), “Exchange of monooleoylphosphatidylcholine as monomer and micelle with membranes containing poly(ethylene glycol)-lipid,” Biophys J 73(5): 2615-29.; Kong, G., G. Anyarambhatla, et al. (2000), “Efficacy of liposomes and hyperthermia in a human tumor xenograft model: importance of triggered drug release,” Cancer Res 60(24): 6950-7.; Matteucci, M. L., G. Anyarambhatla, et al. (2000), “Hyperthermia increases accumulation of technetium-99m-labeled liposomes in feline sarcomas,” Clin Cancer Res 6(9): 3748-55.; Needham, D. and M. W. Dewhirst (2001), “The development and testing of a new temperature-sensitive drug delivery system for the treatment of solid tumors,” Adv Drug Deliv Rev 53(3): 285-305.). These vehicles provide a means for releasing drug in response to heat, however they are inherently leaky in the absence of heat and are limited in the molecular weight of drug that may be loaded within the vehicle.

The vehicles currently in clinical trials load the drug inside a liposome (see e.g. Dewhirst and Needham et al., supra) wherein the outer shell uses a lipid with a phase transition temperature near 42 degrees (DPPC-86 mole percent) in combination with a single acyl chain lipid to create small defects (MPPC-10 mole percent) and a PEGylated lipid (DSPE-PEG 4 mole percent). This combination has been loaded with doxorubicin in clinical trials and shown to have a high systemic toxicity and therefore limited effectiveness.

### SUMMARY

The present invention addresses the above and other limitations of the prior art by providing vehicles that couple an agent to a surface of the vehicle with a cleavable linker.

Accordingly, one aspect of the invention includes compositions including vehicles including one or more layers entrapping a liquid or a solid, wherein the one or more layers comprise an interior surface and an exterior surface, and wherein the interior surface contacts the entrapped liquid, solid, or combination thereof. In an aspect, the vehicles are liposomes. In one aspect, the liposomes are coupled to an agent with a cleavable linker. In another aspect, the liposomes are coupled to a cleaving molecule. In another aspect, the cleavable linker and the cleaving molecule are present on different domains of the exterior surface at a solid or gel phase temperature. In another aspect, the domains of the exterior surface are mixed by raising the temperature of the composition to at least a phase transition temperature. In this aspect, the phase transition accompanying the temperature rise allows the solid or gel phase to convert to a fluid phase. In yet another aspect, the raising of the temperature allows the cleaving molecule to cleave the cleavable linker. In a preferred embodiment, the agent is released from the composition at a desired location by raising the temperature to at least the phase transition temperature. In a related aspect, the phase transition temperature can range from 38 degrees C. to 80 degrees C.

In one embodiment, the surfaces of the liposome includes phospholipids. In one aspect, the cleaving molecule and the cleavable linker are coupled to the phospholipids. In a related aspect, the cleaving molecule is an enzyme. In another related aspect, the cleavable linker is a substrate. In yet another related aspect, the agent coupled to the cleavable linker is a therapeutic agent.

In another embodiment, the composition includes distinct liposomes each coupled to a distinct agent and each further including a distinct phase transition temperature.

In another embodiment, a vehicle of the invention does not include a fatty-acyl peptide, a vehicle is not a microbubble, a vehicle does not include a gas, and/or pressure does not cause mixing of the multiple distinct domains.

The invention also provides a method for treating a subject with the compositions. In one aspect, the composition is administered to the subject. In another aspect, the composition is allowed to accumulate at a site to be targeted with a heating device for a time period. In a related aspect, the targeted site is heated with the heating device and the agents are released from the composition. In one aspect, the time period is 12 hours to 24 hours, 12 hours to 23 hours, 12 hours to 22 hours, 12 hours to 21 hours, 12 hours to 20 hours, 12 hours to 19 hours, 12 hours to 18 hours, 12 hours to 17 hours, 12 hours to 16 hours, 12 hours to 15 hours, 12 hours to 14 hours, 12 hours to 13 hours, 12 hours, or more than 24 hours.

The invention also provides a device for release of the agents from the compositions with heat. In a preferred embodiment, the device includes a temperature feedback device. In another preferred embodiment, the device includes an acoustic pressure feedback device. In yet another preferred embodiment, the temperature-feedback device and the acoustic pressure feedback device are housed in a housing. In one aspect, the housing is a needle or catheter. In another aspect, the invention includes methods for controlling tissue temperature with the device. In a related aspect, the device is inserted into a subject. In another related aspect, the signals from the temperature-feedback device and the acoustic pressure feedback device are coupled. In another related aspect, the coupled signals are used to adjust the parameters of the device for controlling temperature of the tissue.

The invention also provides a composition including one or more layers entrapping a liquid, a solid, or a combination thereof, wherein the one or more layers comprise an interior surface and an exterior surface, and wherein the interior surface contacts the entrapped liquid, solid, or combination thereof; an agent coupled to the exterior surface via a cleavable linker; and a cleaving molecule coupled to the interior surface, wherein the cleavable linker is substantially present on the exterior surface at a first temperature, wherein the cleaving molecule is substantially present on the interior surface at the first temperature, and wherein the surfaces are capable of mixing upon heating of the composition to a second temperature greater than the first temperature.

In one aspect, the vehicle does not include a fatty-acyl peptide, is not a microbubble, does not include a gas, and pressure does not cause mixing of the surfaces of the vehicle.

In one aspect, the cleavable linker includes a disulfide. In one aspect, the cleaving molecule includes a thiol. In one aspect, the first temperature is 37° C. and the second temperature is 42° C.

The invention also provides a composition including a vehicle including one or more layers entrapping a liquid or a solid, wherein the one or more layers comprise an interior surface and an exterior surface, and wherein the interior surface contacts the entrapped liquid, solid, or combination thereof; an agent coupled to the interior surface via a cleavable linker; and a cleaving molecule coupled to the exterior surface, wherein the cleavable linker is substantially present on the interior surface at a first temperature, wherein the cleaving molecule is substantially present on the exterior surface at the first temperature, and wherein the surfaces are capable of mixing upon heating of the composition to a second temperature greater than the first temperature.

In one aspect, the vehicle does not include a fatty-acyl peptide, is not a microbubble, does not include a gas, and pressure does not cause mixing of the surfaces of the vehicle.

In one aspect, the cleavable linker includes a disulfide. In one aspect, the cleaving molecule includes a thiol. In one aspect, the first temperature is 37° C. and the second temperature is 42° C.

The invention also provides a method for treating a subject including administering a composition described above to the subject; allowing the composition to accumulate in a target site of the subject for a time period; heating the target site to the second temperature with a heating device; and releasing the agent from the composition, wherein the agent is released by the cleavage of the cleavable linker by the cleaving molecule.

In one aspect, the time period is 12 hours to 24 hours, 12 hours to 23 hours, 12 hours to 22 hours, 12 hours to 21 hours, 12 hours to 20 hours, 12 hours to 19 hours, 12 hours to 18 hours, 12 hours to 17 hours, 12 hours to 16 hours, 12 hours to 15 hours, 12 hours to 14 hours, 12 hours to 13 hours, 12 hours, or more than 24 hours.

In one aspect of the method, the allowing step is performed after the administering step, the heating step is performed after the allowing step or the administering step, and the releasing step is performed after the heating step. In another aspect of the method the administering step is performed after the heating step.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is an image of phase transition temperature domains that are formed when lipid particles are cooled slowly with lipid separating as a function of phase transition temperature (top images). When cooled rapidly, a homogenous particle surface is formed (bottom images). Images shown are of monolayers of lipid with a diameter of approximately 10 micrometers. Heating particles with discrete domains (top images) results in lipid “mixing” and mobility-based release of agent.

FIG. 2 illustrates one embodiment of the mobility-based release mechanism. Reaction of a thiol and N-[3-(2-Pyridyldithio) propionyl]-1,2-dipalmitoyl-sn-glycero-3-phosphoethanolamine (PDP-PE) releases a chromophore.

FIG. 3 illustrates a liposome with a lipid bilayer shell comprising head groups and tails and shows that agent, linker, and enzyme can be attached to the head group of either the interior or the exterior surface of the liposome shell.

FIG. 4 is a schematic of an ultrasound heating device.

FIG. 5 illustrates a proportional-integral-differential (PID) algorithm.

FIG. 6 is a screen shot that illustrates an example of a PID controlled heating profile.

FIG. 7 illustrates one embodiment of an illustration of thermocouple bracket design.

FIG. 8 illustrates another embodiment of an illustration of thermocouple bracket design.

FIG. 9 illustrates in vivo results demonstrating of ultrasound heating and release of liposome contents.

FIG. 10 is a schematic of the synthesis of PDP-PE.

FIG. 11 is a schematic of the synthesis of N-Succinimidyl S-Acetylthioacetate (SATA)-1,2-distearoyl-sn-Glycero-3-phosphoethanolamine (DSPE).

FIG. 12 is a schematic of the synthesis of N-Succinimidyl S-Acetylthiopropionate (SATP)-DSPE.

FIG. 13 is a schematic of the thiolation of DSPE.

FIG. 14 provides UV-Vis spectra of one embodiment of the vehicles of the present invention, liposomes with a composition of DPPC 80%, DSPE-PEG2000 2%, DSPE 9%, and 16:0 PDP-PE 9% that was reacted with 2-IT (2-iminothiolane) and purified. The first 90 C liposomes legend refers to uv-vis spectra collected after heating the purified liposomes to 90 C for 10 min and then cooling to room temperature. The second 90 C liposomes legend was a repeat of heating the liposomes to 90C for an extra 10 minutes. The borate blank was a spectrum collected of the borate buffer in which the experiments were performed.

FIG. 15 provides UV-Vis spectra of a second embodiment of the vehicles of the present invention.

FIG. 16 provides UV-Vis spectra for negative control of a second embodiment of the vehicles of the present invention with protected liposomes.

FIG. 17 provides UV-Vis spectra illustrating a test for total SATA-DSPE in liposomes of a second embodiment of the vehicles of the present invention.

FIG. 18 provides UV-Vis spectra illustrating a test for effect of temperature and hydroxylamine on DTP of a second embodiment of the vehicles of the present invention.

FIG. 19 provides UV-Vis spectra illustrating a test for effect of heat with no hydroxylamine solution on disulfide bonds of a second embodiment of the vehicles of the present invention.

FIG. 20 provides UV-Vis spectra illustrating a third embodiment of the vehicles of the present invention.

FIG. 21 provides UV-Vis spectra for a negative control for a third embodiment of the vehicles of the present invention with protected liposomes.

FIG. 22 provides UV-Vis spectra illustrating a test for effect of heat alone of a third embodiment of the vehicles of the present invention.

FIG. 23 provides UV-Vis spectra illustrating a test for effect of heat and hydroxylamine of a third embodiment of the vehicles of the present invention.

FIG. 24 shows the mechanism of flip-flop release from liposomes. At top, liposomes are formed with lipids containing a pyridyl disulfide functional group present on both outside and inside layers. Tris (2-carboxyethyl) phosphine (TCEP) selectively reduces the disulfide functional group on the outside layer while preserving the disulfides on the inside of the liposome. Bottom left—At the melting temperature of 1,2-dipalmitoyl-sn-glycero-3-phosphocholine (DPPC), the lipids will undergo a flip-flop and switch layers. When the lipids are present in the same layer, a thiol-disulfide reaction will occur and will release pyridine-2-thione.

FIG. 25 shows the chemical structure of 1,2-dipalmitoyl-sn-glycero-3-phosphoethanolamine-N-[3-(2-pyridyldithio) propionate (16:0 PDP-PE).

FIG. 26 shows the chemical structure of 1,2-distearoyl-sn-glycero-3-phosphoethanolamine-N-[3-(2-pyridyldithio) propionate (18:0 PDP-PE).

FIG. 27 shows the chemical structure of DTP-21T-DPPE.

FIG. 28 shows chromophore release from liposomes, the composition is shown in Table 1, at 42° C., 37° C., and 25° C.

FIG. 29 shows chromophore release from liposomes, the composition is shown in Table 2, at 42° C., 39° C., and 37° C.

FIG. 30 shows chromophore release from liposomes, the composition is shown in Table 3, at 55° C., 47° C., and 42° C.

FIG. 31 shows chromophore release from liposomes, the composition is shown in Table 4, at 42° C., 39° C., and 37° C.

FIG. 32 shows chromophore release from liposomes, the composition is shown in Table 5, at 42° C., 39° C., and 37° C. The counter ion to the probe lipid was sodium. The probe lipid was synthesized separately from triethylammonium analogue used in FIG. 33.

FIG. 33 shows chromophore release from liposomes, the composition is shown in Table 5, at 42° C. and 37° C. Counter ion to the probe lipid was triethylammonium. Probe lipid was synthesized separately from sodium analogue used in FIG. 32.

FIG. 34 shows chromophore release from liposomes, the composition is shown in Table 6, at 42° C., 39° C., and 37° C.

FIG. 35 shows chromophore release from liposomes, the composition is shown in Table 7, at 42° C., 39° C., and 37° C.

FIG. 36 shows chromophore release from liposomes, the composition shown in Table 8, at 42° C., 39° C., and 37° C.

FIG. 37 shows chromophore release from liposomes, the composition is shown in Table 9, at 42° C. and 37° C.

FIG. 38 shows side by side comparison of FIGS. 28 and 30. Effect of liposome melting temperature on flip-flop rate. DS in legend next to the experiments specified temperature refers to 1,2-distearoyl-sn-glycero-3-phosphocholine (DSPC) containing liposomes, DP refers to DPPC containing liposomes.

FIG. 39 shows side by side comparison of FIGS. 34 and 35. Liposomes contained equal molar amounts of DPPC and DSPC, where (1) in legend refers to liposomes containing 18:0 PDP-PE (Table 7) and (2) refers to liposomes containing 16:0 PDP-PE (Table 6).

FIG. 40 shows side by side comparison of FIGS. 32 and 33 (sodium and triethylammonium analogues of Table 5). Tea in legend refers to triethylammonium counter ion to probe lipid, sodium refers to the sodium adduct of the probe lipid.

FIG. 41 shows side by side comparison of FIGS. 28 and 37. DPPC liposomes containing 20% cholesterol are referred to as DPPC Chol in legend. Liposomes that contained no cholesterol are labeled as DPPC in legend.

FIG. 42 shows side by side comparison of FIGS. 32 and 36. Temperature with (2) in legend refers to liposome composition from Table 5. Temperatures alone refer to liposomes composition from Table 8.

FIG. 43 shows examples of head groups useful in the invention.

### DETAILED DESCRIPTION

### Advantages and Utility

Briefly, and as described in more detail below, described herein are methods, compositions, and device for releasing an agent in a controlled and directed manner with heat.

Advantages of this approach are numerous. Among the advantages is improved specificity and reduced toxicity for administered compounds, and improved treatment outcomes for subjects in need of treatment for a wide variety of medical conditions, especially cancers, cardiovascular diseases, and inflammatory disorders such as rheumatoid arthritis and Crohn's disease.

The invention is useful for diagnostic and or therapeutic applications in which it is beneficial to administer an agent such as, e.g., a physiologically-active agent, for the purpose of imaging, diagnosing and/or treating a medical condition.

### DEFINITIONS

Terms used in the claims and specification are defined as set forth below unless otherwise specified.

The term “subject” as used herein includes both humans and non-humans and includes but is not limited to humans, non-human primates, canines, felines, murines, bovines, equines, and porcines.

The term “cleavable linker” as used herein refers to any first molecule coupled to a vehicle membrane that can be cleaved by any second molecule when the vehicle membrane is in a fluid phase. Examples of preferred cleavable linkers are listed below.

The term “cleaving molecule” as used herein refers to any first molecule coupled to a vehicle membrane that can cleave any second molecule when the vehicle membrane is in a fluid phase. Examples of preferred cleaving molecules are listed below.

The term “domain” as used herein refers to a first region of a vehicle membrane that is distinct from a second region of the vehicle membrane that is in a non-fluid phase.

The term “vehicle” refers to any particle with a shell material and a lipid bilayer; such particles are, e.g., liposomes and micelles.

The term “mixing” refers to two or more distinct components of a vehicle, located at at least a first position and a second position, moving to a proximate position upon application of heat to the vehicle. For example, “mixing” can refer to the ability of a cleaving molecule attached to a first component at a first position to move to a second position in or on the vehicle upon heating of the vehicle, where the movement of the first component to the second position allows the cleaving molecule to interact with a cleavable linker attached to a second component of the vehicle. As another example, “mixing” can also include a cleavable linker passing from the interior surface of a vehicle to the exterior surface of the vehicle upon heating. Examples of distinct components of a vehicle are an inner shell, an outer shell, a surface of a layer of the vehicle, and/or distinct domains of a layer.

The “direction” of ultrasound pulses for tissue heating may consist of the following: a) human or any intelligent or automated delineation or specification of the region to be heated, or region-of-interest (ROI); b) human or any intelligent or automated control of the acoustic energy delivered to a specified ROI.

“Heating tissue” is the result of thermal energy deposition due to viscous losses associated with the propagation of ultrasound (longitudinal wave) through tissue (a visco-elastic medium).

A “transducer” is any device that converts electrical energy to mechanical energy in the form of longitudinal ultrasound waves and vice versa.

“Controlling duty-cycle” refers to modifying pulse-length and/or pulse-repetition-frequency (PRF) or any means by which the ratio of “on” time to the total “on” plus “off” time is modified. For example, the PRF for tissue heating pulses may not be constant if imaging pulse sequences are interleaved.

“Feedback” may take the form of some combination of hardware and software feedback. The feedback may be “real-time” in the sense that there the feedback control algorithm calculates in less time than the sampling period of the temperature signal. If a stand-alone ultrasound system is modified, the feedback may involve communicating control signals or commands to the system (e.g., using an Ethernet connection, proportional analog input, and/or dedicated digital logic).

It must be noted that, as used in the specification and the appended claims, the singular forms “a,” “an,” and “the” include plural referents unless the context clearly dictates otherwise.

### DESCRIPTION

The present invention provides vehicles coupled to a cleavable linker. Preferably, the vehicles are liposomes. In one aspect, the cleavable linker allows the release of an agent attached to the linker at a target site in a subject's body that is subjected to an elevation of temperature of the subject's body compared to the normal temperature of the subject As an example, liposomes of the present invention are particularly useful in drug delivery, where the liposome is coupled to a compound to be delivered to a preselected target site in a subject's body. The target site may be artificially heated by, e.g., hyperthermia so that it is at or above the phase transition temperature of the vehicle. Preferably, the compound is released at the preselected target site once the phase transition temperature of the vehicle is reached. The present invention also provides a device for use with vehicles of the present invention and other vehicles known in the art.

Vehicles

It should be appreciated that membrane-forming material of a vehicle can be any lipid comprising material that is sensitive to a change in temperature. Preferably, membrane-forming material responds to a change in temperature by changing phase or state i.e., is a temperature-sensitive material. Exemplary materials which may form a solid-phase membrane include, but are not limited to, natural lipids, synthetic lipids, phospholipids, or microbial lipids. The above noted materials are examples of a layer, inner shell and/or outer shell materials of the vehicles of the present invention.

The use of lipid formulations is contemplated for the introduction of an agent. In a specific embodiment of the invention, the agent may be associated with a lipid. The agent associated with a lipid may be attached to a liposome via a linking molecule that is associated with both the liposome and the agent. The linking molecule is preferably cleavable. More preferably, the linking molecule is cleavable in response to an increase in temperature, due to heating. The lipid-agent compositions of the present invention are not limited to any particular structure in solution. For example, they may be present in a bilayer structure, as micelles, or with a “collapsed” structure. They may also simply be interspersed in a solution, possibly forming aggregates which are not uniform in either size or shape.

Lipids are fatty substances which may be naturally occurring or synthetic. For example, lipids include the fatty droplets that naturally occur in the cytoplasm as well as the class of compounds which are well known to those of skill in the art that contain long-chain aliphatic hydrocarbons and their derivatives, such as fatty acids, alcohols, amines, amino alcohols, and aldehydes. Additional examples of suitable lipids include hydrogenated lecithin from plants and animals, such as egg yolk lecithin and soybean lecithin. The lipid can also be phosphatidyl choline produced from partial or complete synthesis containing mixed acyl groups of lauryl, myristoyl, palmitoyl and stearoyl.

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid layers. However, the present invention also encompasses compositions that have different structures in solution than the normal vesicular structure. For example, the lipids may assume a micellar structure or merely exist as nonuniform aggregates of lipid molecules. Also contemplated are lipofectamine-agent complexes. The liposome is one example of a vehicle with an inner shell and an outer shell of the present invention.

A neutrally charged lipid can comprise a lipid with no charge, a substantially uncharged lipid, or a lipid mixture with equal number of positive and negative charges. Suitable phospholipids include phosphatidyl cholines and others that are well known to those of skill in the art.

Phospholipids may be used for preparing the liposomes according to the present invention and may carry a net positive, negative, or neutral charge. For example, diacetyl phosphate can be employed to confer a negative charge on the liposomes, and stearylamine can be used to confer a positive charge on the liposomes. The liposomes can be made of one or more phospholipids.

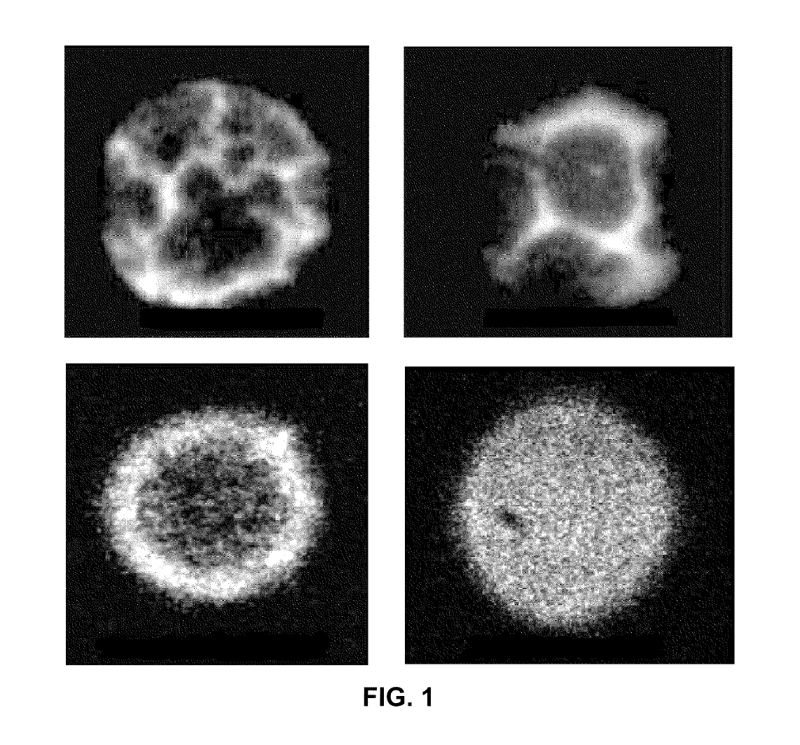

Phospholipids can form a variety of structures other than liposomes when dispersed in water, depending on the molar ratio of lipid to water. At low ratios the liposome is the preferred structure. The physical characteristics of liposomes depend on pH, ionic strength, and/or the presence of divalent cations. Liposomes can show low permeability to ionic and/or polar substances, but at elevated temperatures undergo a “phase transition” which markedly alters their permeability. The phase transition involves a change from a closely packed, ordered structure, known as the gel phase, to a loosely packed, less-ordered structure, known as the fluid phase. This occurs at a characteristic phase-transition temperature, such as e.g. 37-45° C., and/or results in an increase in permeability to ions, sugars, and/or drugs. The gel phase is an ordered arrangement of the phospholipids, where the fatty acid chains are locked in staggered conformations or “domains,” which result in minimal interactions of different phospholipids in a membrane, as shown in FIG. 1. This is one example of distinct domains according to the present invention. The fluid phase is characterized by a random arrangement of the phospholipids in a membrane. The different factors that influence a particular lipid's transition temperature can include, e.g., the number of carbons in the fatty acid chains, the number of double bonds present, type of head-group present, and the overall charge of the molecule. By controlling the ratios of phospholipids, each with different phase transition temperatures, the degree of interaction between molecules can be regulated by controlling the temperature.

The phase transition temperature of the phospholipid is selected to control the temperature that the domains mix and the agent is released from the liposomes. Phospholipids are known to have different phase transition temperatures and can be used to produce liposomes having release temperatures corresponding to the phase transition temperature of the phospholipids. Suitable phospholipids include, for example, dimyristoylphosphatidyl choline having a phase transition temperature of 23.9° C., palmitoylmyristoylphosphatidyl choline having a phase transition temperature of 27.2° C., myristolypalmitoylphosphatidyl choline having a phase transition temperature of 35.3° C., dipalmitoylphosphatidyl choline having a phase transition temperature of 41.4° C., stearoylpalmitoylphosphatidyl choline having a phase transition temperature of 44.0° C., palmitoylstearolyphosphatidyl choline having a phase transition of 47.4° C., and distearolyphosphatidyl choline having a phase transition temperature of 54.9° C. Another suitable phospholipid is a synthetic C₁₇ phosphatidyl choline from Aventi Inc. having a phase transition temperature of about 48-49° C.

The phase transition temperature of the liposomes can be selected by combining the different phospholipids during the production of the liposomes according to the respective phase transition temperature. The phase transition of the resulting liposome membrane is generally proportional to the ratio by weight of the individual phospholipids. Thus, the composition of the phospholipids is selected based on the respective phase transition temperature so that the phase transition temperature of the liposome membrane will fall within the selected range. By adjusting the phase transition temperature of the liposome membrane to the selected range, the temperature at which the liposomes release the agents can be controlled during heating.

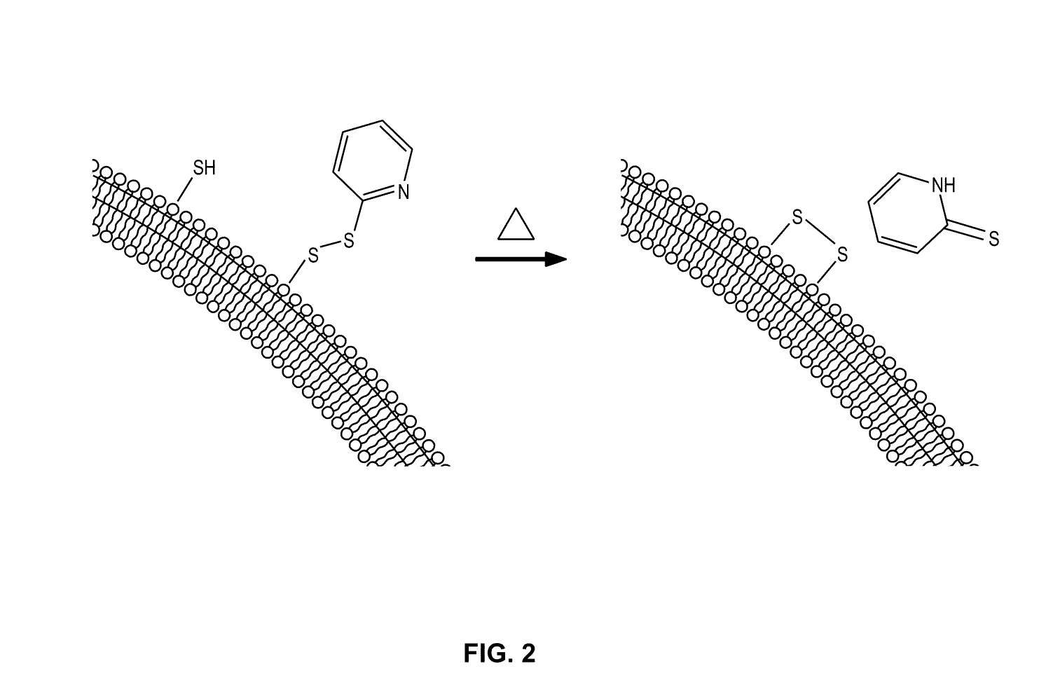

In one embodiment of the present invention, the phospholipid phosphotidylethanolamine is used, which contains an amine group that allows for chemical conjugation. In another example of the present invention, the liposomes are formulated with mostly dipalmitoylphosphatidyl choline (DPPC), which has a phase transition temperature of about 42° C., and a small amount of two reactive phospholipids. Thus, below 42° C., the two reactive species will not interact significantly, and when the liposomes are warmed above this phase transition temperature, a reaction will occur, as shown in FIG. 2. For example, a quenched chromophore is connected to a synthetic phospholipid of a vehicle by a disulfide bond, upon heating a reaction will occur between the disulfide bond and a thiol connected to a synthetic phospholipid of the vehicle, thus releasing the chromophore from the vehicle. This is one example of a releasing method of the present invention. A disulfide bond is one example of a cleavable linker of the present invention. A thiol is one example of a cleaving molecule of the present invention. The other phospholipid may contain a protected thiol, which upon treatment with hydroxylamine will form the thiol. The phase transition temperature of the instant invention can preferably range from 38° C. to 80° C., depending on the molecular composition of the preferred vehicle. More preferably the phase transition temperature can range from 38° C. to 50° C. More preferably the phase transition temperature can range from 39° C. to 45° C. More preferably the phase transition temperature is 42° C.

In another embodiment of the invention, the composition contains a mixture of liposomes having different phase transition temperatures to release the agents at different temperatures. In one embodiment, the liposome composition contains liposomes coupled to a first agent and having a phase transition temperature of 42° C. to about 45° C. and liposomes coupled to a second agent and having a phase transition temperature of about 50° C. or higher. In one embodiment, the second agent is coupled to a liposome that releases the agent at a temperature range of 50° C. to 60° C. In this embodiment, the liposome composition is delivered to the target and the target site is subjected to hyperthermal (i.e., above normally-occurring) temperatures. As the tissue in the target site is heated to at least 42° C., the first liposomes release the first agent. In preferred embodiments of the invention, the hyperthermal treatment does not exceed a temperature sufficient to cause protein denaturization. In this embodiment, the second liposomes are selected to release the second agent at or slightly below the protein denaturization temperature. This embodiment allows a user to release a combination of drugs at a target site in a subject.

In another embodiment, the composition can contain several liposomes that can transition at different temperatures to release a plurality of agents at incremental temperatures as the temperature of the target site increases. In one embodiment, the liposomes can be selected to release agents at 2° C. intervals between about 42° C. and 50° C. The agents for each liposome can be different.

Liposomes interact with cells via four different mechanisms: Endocytosis by phagocytic cells of the reticuloendothelial system such as macrophages and/or neutrophils; adsorption to the cell surface, either by nonspecific weak hydrophobic and/or electrostatic forces, and/or by specific interactions with cell-surface components; fusion with the plasma cell membrane by insertion of the lipid bilayer of the liposome into the plasma membrane, with simultaneous release of liposomal contents into the cytoplasm; and/or by transfer of liposomal lipids to cellular and/or subcellular membranes, and/or vice versa, without any association of the liposome contents. Varying the liposome formulation can alter which mechanism is operative, although more than one may operate at the same time.

Liposomes used according to the present invention can be made by different methods known to those of ordinary skill in the art. The size of the liposomes varies depending on the method of synthesis. Preferably, liposomes are from about 1 nm, 10 nm, 50 nm, 100 nm, 120 nm, 130 nm, 140 nm, or 150 nm, up to about 175 nm, 180 nm, 200 nm, 250 nm, 300 nm, 350 nm, 400 nm, 500 nm, 1 μm, 10 μm, 100 μm, or 1000 μm in diameter. A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and outside the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

Liposomes within the scope of the present invention can be prepared in accordance with known laboratory techniques. In one preferred embodiment, liposomes are prepared by mixing liposomal lipids, in a solvent in a container, e.g., a glass, pear-shaped flask. The container may have a volume ten-times greater than the volume of the expected suspension of liposomes. Using a rotary evaporator, the solvent is removed at approximately 40° C. under negative pressure. The solvent normally is removed within about 5 mM to 2 hours, depending on the desired volume of the liposomes. The composition can be dried further in a desiccator under vacuum. The dried lipids generally are discarded after about 1 week because of a tendency to deteriorate with time.

Dried lipids can be hydrated at, e.g., approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In the alternative, liposomes can be prepared in accordance with other known laboratory procedures: the method of Bangham et al. (1965), the contents of which are incorporated herein by reference; the method of Gregoriadis, as described in Drug Carriers in Biology and Medicine, G. Gregoriadis ed. (1979) pp. 287-341, the contents of which are incorporated herein by reference; the method of Deamer and Uster, 1983, the contents of which are incorporated by reference; and the reverse-phase evaporation method as described by Szoka and Papahadjopoulos, 1978. The aforementioned methods differ in their respective abilities to entrap aqueous material and their respective aqueous space-to-lipid ratios.

The dried lipids or lyophilized liposomes prepared as described above may be dehydrated and reconstituted in a solution of inhibitory peptide and diluted to an appropriate concentration with a suitable solvent. The mixture is then vigorously shaken in a vortex mixer. Contaminates are removed by centrifugation at 29,000×g and the liposomal pellets washed. The washed liposomes are resuspended at an appropriate total phospholipid concentration, e.g., about 50-200 mM.

Micelles within the scope of the present invention can be prepared in accordance with known laboratory techniques. Preferably, micelles can be prepared in accordance with the methods of: J. M. Seddon, R. H. Templer. Polymorphism of Lipid-Water Systems, from the Handbook of Biological Physics, Vol. 1, ed. R. Lipowsky, and E. Sackmann. (c) 1995, Elsevier Science B.V. ISBN 0-444-81975-4., the contents of which are incorporated by reference; S. A. Baeurle, J. Kroener, Modeling effective interactions of micellar aggregates of ionic surfactants with the Gauss-Core potential, J. Math. Chem. 36, 409-421 (2004)., the contents of which are incorporated by reference; McBain, J. W., Trans. Faraday Soc. 1913, 9, 99., the contents of which are incorporated by reference; Hartley, G. S., Aqueous Solutions of Paraffin Chain Salts, A Study in Micelle Formation, 1936, Hermann et Cie, Paris., the contents of which are incorporated by reference.

Agents

Agents suitable for use in the present invention include therapeutic agents and pharmacologically active agents, nutritional molecules, cosmetic agents, diagnostic agents and contrast agents for imaging. Agents may also include nucleic acids, e.g., genes, siRNA, microRNA, vectors, or gene fragments. As used herein, agent includes pharmacologically acceptable salts of agents. Suitable therapeutic agents include, for example, antineoplastics, monomethylauristatin E (MMAE), monomethylauristatin F (MMAF), antitumor agents, antibiotics, antifungals, anti-inflammatory agents, immunosuppressive agents, anti-infective agents, antivirals, anthelminthic, and antiparasitic compounds. Suitable antitumor agents include agents such as cisplatin, carboplatin, tetraplatin and iproplatin. Suitable antitumor agents also include adriamycin, mitomycin C, actinomycin, ansamitocin and its derivatives, bleomycin, Ara-C, doxorubicin, daunomycin, metabolic antagonists such as 5-FU, methotrexate, isobutyl 5-fluoro-6-E-furfurylideneamino-xy-1,2,3,4,5,6 hexahydro-2,4-dioxopyrimidine-5-carboxylate. Other antitumor agents include melpharan, mitoxantrone and lymphokines. The amount of the particular agent coupled to the liposome is selected according to the desired therapeutic dose and/or the unit dose.

Heat

The present invention provides vehicles that release coupled contents at temperatures that can be achieved in clinical settings using heat such as mild hyperthermia. As used herein, the term “hyperthermia” refers to the elevation of the temperature of a subject's body, or a part of a subject's body, compared to the normal temperature of the subject. Conditions for mild hyperthermia typically range from 37 to 42° C. (Murata, R. and M. R. Horsman (2004). “Tumour-specific enhancement of thermoradiotherapy at mild temperatures by the vascular targeting agent 5,6-dimethylxanthenone-4-acetic acid.” Int J Hyperthermia 20(4): 393-404.; Horsman, M. R. (2006). “Tissue physiology and the response to heat.” Int J Hyperthermia 22(3): 197-203.; Li, G. C., F. He, et al. (2006). “Hyperthermia and gene therapy: potential use of microPET imaging.” Int J Hyperthermia 22(3): 215-21.; Myerson, R. J., A. K. Singh, et al. (2006). “Monitoring the effect of mild hyperthermia on tumour hypoxia by Cu-ATSM PET scanning” Int J Hyperthermia 22(2): 93-115.). Mild hyperthermia causes several physiological effects including, but not limited to increased blood flow, increased oxygenation, increased microvascular permeability, increased pH, increased heat shock protein production, and decreased healing time for musculo-skeletal injuries. It has been demonstrated that mild hyperthermia increases the effectiveness of radiochemotherapy in human tumors. It has also been demonstrated that mild hyperthermia increases vascular permeability to allow extravasation of nanoparticles and molecules including but not limited to albumin, dextran, liposomes, micelles, quantum dots, and polymers. Heat for hyperthermia can be produced by, e.g., irradiation with acoustic waves, electromagnetic waves, ionizing radiation, laser irradiation, microwaves.

Heat for use with the vehicles of the present invention can be applied using any heating device known in the art or later discovered. For example, the heating device preferably includes a suitable heat or energy source that is able to focus the heat or energy on the target and is able to control heat and temperature of the tissue. The heat source can be an electrical resistance heating element, or an indirectly heated element. The heating device can also have an energy source for producing heat at the target site, such as a radio frequency (“RF”) device, ultrasonic generators, laser, or infrared device. One example of an RF generator heating device for hyperthermally treating tissue in a selected target site is disclosed in U.S. Pat. No. 6,197,022, which is hereby incorporated by reference in its entirety. Examples of suitable ultrasound heating devices for delivering ultrasonic hyperthermia are disclosed in U.S. Pat. Nos. 4,620,546, 4,658,828 and 4,586,512, the disclosures of which are hereby incorporated by reference in their entirety. Preferably, heat is applied using an ultrasound device. For example, heat is applied using the ultrasound heating device of the instant invention described below.

The heat source can be applied to a variety of the areas in a body where hyperthermal treatment is desired, such as e.g. a target site. The target site is a localized site or region of the body and can be e.g. tumors, organs, muscles, and soft tissue.

Cleavable Linkers

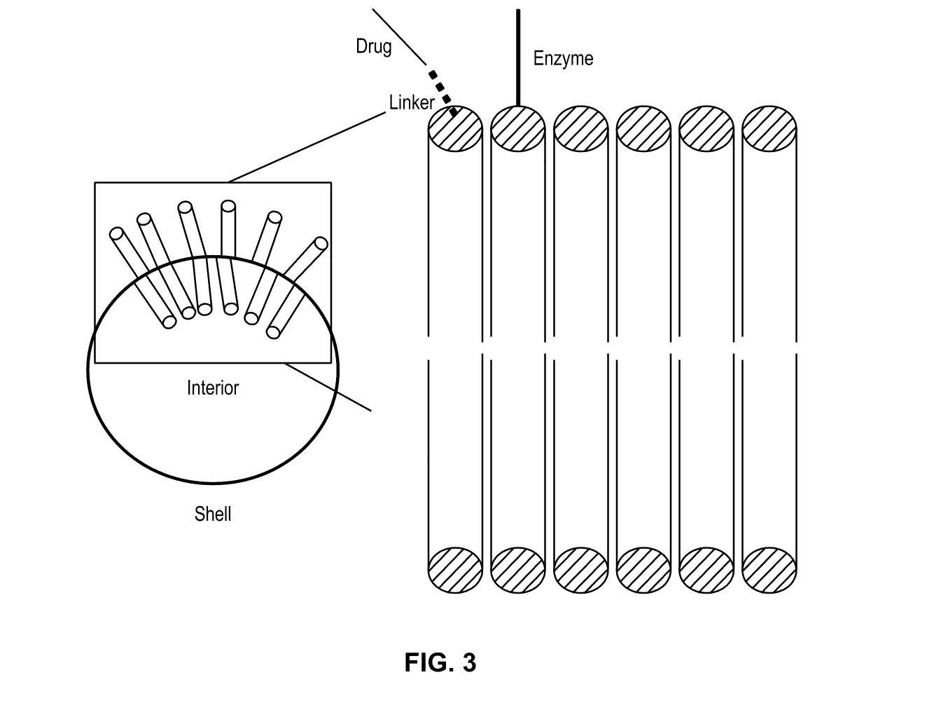

The present invention provides a cleavable linker that couples an agent to a vehicle. The cleavable linker is a molecule that can be cleaved. Cleavable linkers can include, but are not limited to, any peptide, lipid, nucleic acid, or chemical that can be cleaved such as a substrate, a non-human substrate, a non-mammalian substrate, a non-eukaryotic substrate, a disulfide, a disulfide bond, a cathepsin substrate, or N-[3-(2-Pyridyldithio) propionyl]-1,2-dipalmitoyl-sn-glycero-3-phosphoethanolamine (“PDP-PE”). The cleavable linker can be attached to either the head or tail of a lipid, as shown in FIG. 3. Preferably the cleavable linker is resistant to cleavage by human enzymes. More preferably, the cleavable linker is resistant to cleavage by human liver enzymes.

Cleaving Molecules

The present invention provides a cleaving molecule that cleaves the cleavable linker of the present invention. Cleaving molecules can include, but are not limited to, any peptide, lipid, nucleic acid, or chemical that can cleave such as a thiol, an enzyme, a non-human enzyme, a non-mammalian enzyme, a non-eukaryotic enzyme, a peptidase, a protease, cathepsin, an amine, a thioacetate ester, a sulfhydryl group, 1,2-distearoyl-sn-Glycero-3-phosphoethanolamine (DSPE), N-Succinimidyl S-Acetylthioacetate (SATA)-DSPE, N-Succinimidyl S-Acetylthiopropionate (SATP)-DSPE, or SATA-1,2-dipalmitoyl-sn-Glycero-3-phosphoethanolamine (DPPE). It is within the scope of the present invention to use various substances to increase the cleaving activity of the cleaving molecule, such as treatment SATP-DSPE or SATA-DSPE with hydroxylamine. The cleaving molecule can be attached to either the head or tail of a lipid, as shown in FIG. 3. Preferably the cleavable linker is resistant to damage by human enzymes. More preferably, the cleavable linker is resistant to damage by human liver enzymes.

Ultrasound Heating Device

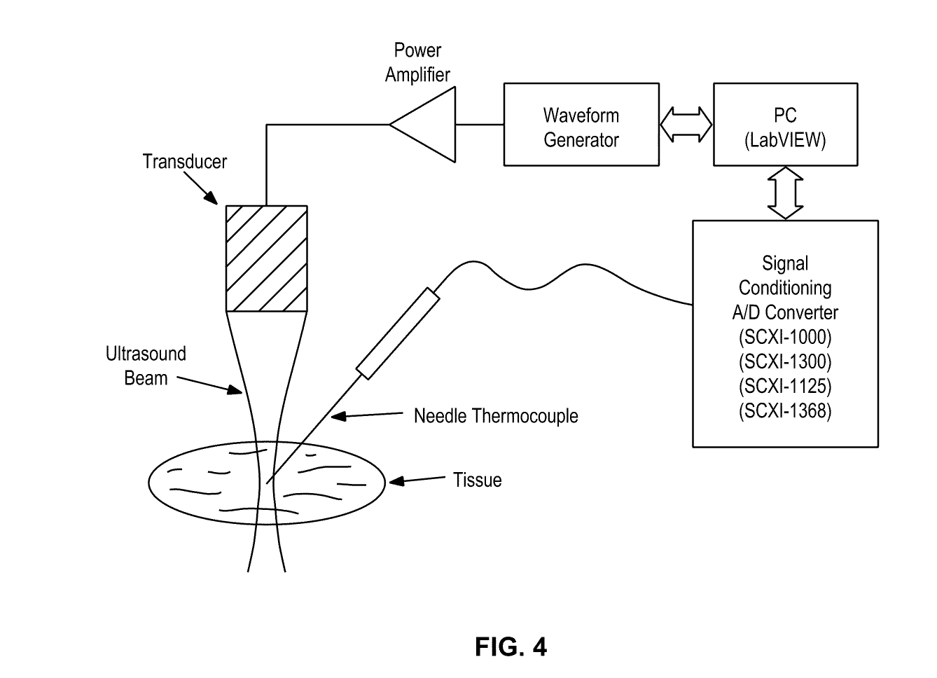

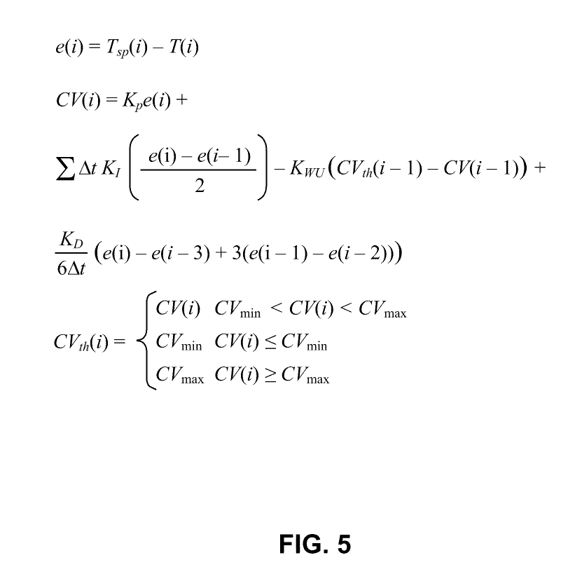

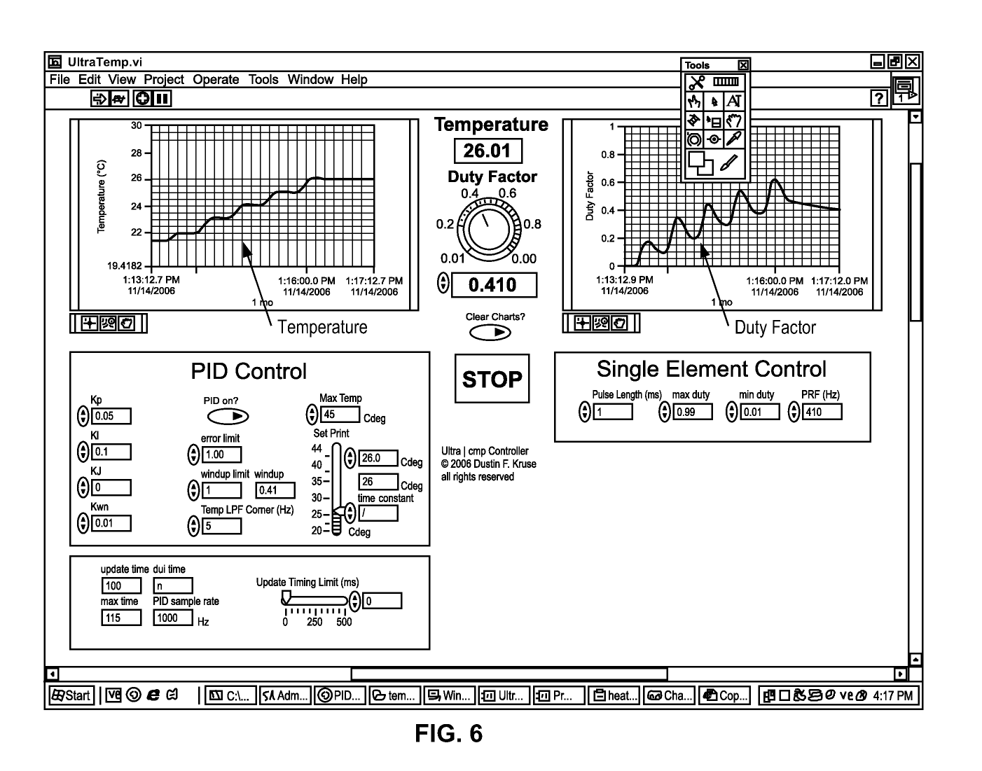

One embodiment of the invention encompasses an ultrasound heating device that is used to heat tissue in a controllable and verifiable way for the purpose of releasing a drug, shifting pH, enhancing uptake of a drug or drug delivery vehicle, oxygenation, and/or general-purpose hyperthermia. FIG. 4 is a block diagram showing one embodiment of such an ultrasound heating device. The depicted embodiment of the ultrasound heating device consists of an ultrasound transducer connected to a power amplifier, which amplifies pulse signals from a connected arbitrary waveform generator. The arbitrary waveform generator generates triggered 1 millisecond duration tone-bursts with variable pulse-repetition-frequency (PRF). A PCI-6602 counter/timer board controlled by a PC running LabVIEW is used to generate the trigger signals. The needle thermocouple was purchased from Physitemp Instruments, Inc., and is a type-T thermocouple inserted at the tip of a 29 gauge hollow, stainless-steel needle. The needle was sealed, and closed off and sharpened at the tip. The junction of the thermocouple was not exposed; only stainless steel contacts the tissue. The thermocouple itself was not electrically insulated. The thermocouple was connected to a signal conditioner (National Instruments, SCXI-1125) using an isothermal terminal block (SCXI-1368), and the conditioned thermocouple signal was sampled at 1 kHz using a 16-bit A/D converter (SCXI-1600). All of the SCXI modules were contained within a SCXI-1000 chassis. The digital samples were communicated to a PC over a USB bus, and software written in LabVIEW further conditions the samples. The conditioned samples were used as feedback in a proportional-integral-differential (PID) loop, as shown in FIG. 5, to control the temperature at the thermocouple by commanding a duty factor that ranges from 0.01 to 0.99. The integral portion of the PID loop had an anti-windup mechanism, as well as an integral wind-up limit, both of which serve to increase the responsiveness of the integral part of the loop and reduce overshoot. Typically, the integral and proportional gains are set in the range of 0.05 to 0.20, and the differential gain is set to zero, but the gains are dependent on the amount of acoustic power available. The PID loop runs at the full sample rate. The PRF for triggering the ultrasound pulse generation was calculated from the duty factor (or duty cycle). The duty factor was updated at approximately 10 Hz, which is mainly limited by the time it takes to update PCI-6602 counter/timer board. The voltage of the waveform input to the power amplified was controlled manually, but it may be controlled through software. FIG. 6 demonstrates an example of a PID-controlled heating profile.

The ultrasound heating device may comprise an ultrasound imaging device capable of optimized ultrasound imaging, and within an imaging exam capable of directing ultrasound pulses for the purpose of heating tissue for the purpose of releasing a drug, shifting pH, enhancing uptake of a drug or drug delivery vehicle, oxygenation, and/or general-purpose hyperthermia treatment. The ultrasound imaging device may include any conventional ultrasound imaging device that is modified to direct ultrasound pulses for the purpose of heating tissue. In one embodiment of the ultrasound imaging device, information from an ultrasound image is used to define the region of interest (ROI) (i.e., the ROI is defined relative to anatomical information in the ultrasound image). In another embodiment of the ultrasound imaging device, a user directs a “heating volume” in an equivalent way that a “sample volume” is directed in a pulsed-Doppler examination. The “heating volume” and “sample volume” refer to the range gated region in a subject targeted by the ultrasound imaging device. In another embodiment of the ultrasound imaging device, the user directs a “heating volume” in an equivalent way that a ROI is directed in a Color-Doppler examination.

The ultrasound heating device may further comprise an ultrasound transducer for both generating high frequency ultrasound pulses for imaging and/or lower frequency ultrasound pulses for tissue heating. An ultrasound transducer that operates over a wide range of frequencies and is able to transmit wide bandwidth pulses and receive wide bandwidth echoes for ultrasound imaging and that is also able produce ultrasound pulses for the purpose of heating tissue is preferred. In another embodiment, the ultrasound transducer has modifications for dissipating thermal energy generated by electrical and acoustic absorption in and adjacent to the piezoelectric element. The transducer may consist of more than one transducer, each being optimized for both imaging and tissue heating. Alternatively, the transducer may consist of more than one transducer, each being optimized for either imaging or tissue heating. In another embodiment of the transducer, the beam originating from the transducer may be mechanically scanned or electronically scanned. Mechanical scanning may be facilitated using an acoustic mirror to reflect the ultrasound beam in different directions. In another embodiment, the transducer may consist of two transducer arrays for tissue heating on either side of a center transducer array used for imaging. This is called a “co-linear” array.

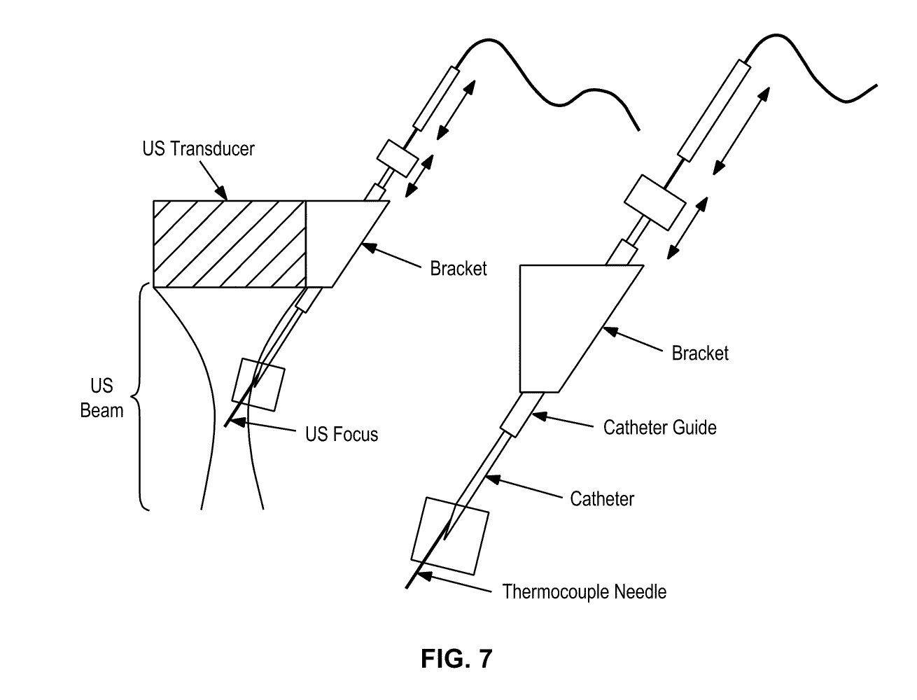

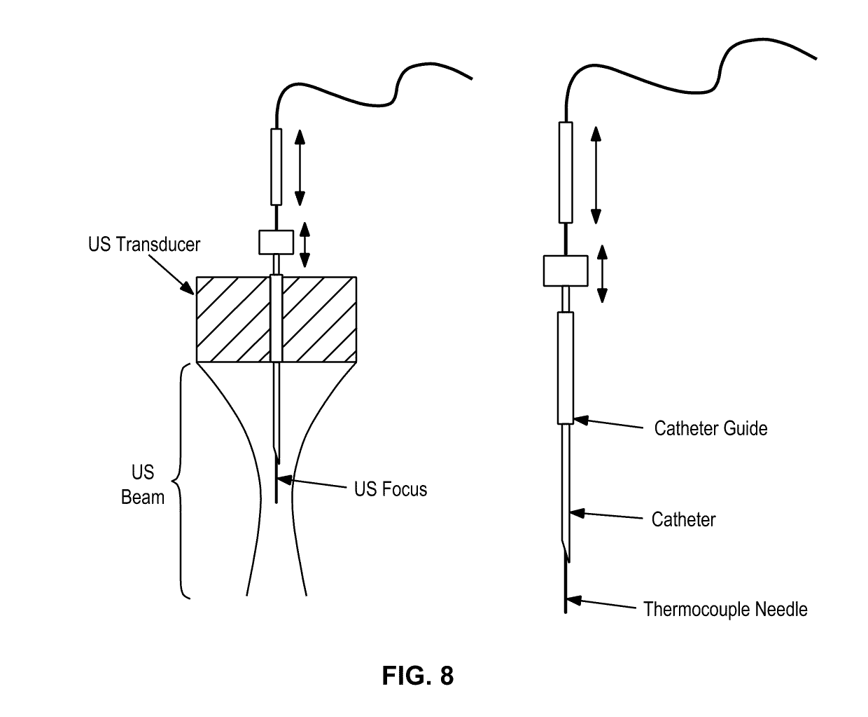

The ultrasound heating device may further comprise a temperature feedback device comprising a thermocouple. Preferably, the temperature feedback device is sealed within a fine gauge stainless steel needle (e.g., 29 gauge) or catheter that is substantially resistant to viscous heating from ultrasound pulses used for tissue heating. More preferably, the temperature feedback device senses temperature. Temperature feedback from the temperature feedback device is important because every tissue has a unique acoustic absorption, thermal properties, convective loss (e.g., blood flow). In one embodiment, the temperature feedback device provides a temperature-dependent signal that is interpretable by the ultrasound imaging device. The temperature-dependent signal may, e.g., allow the ultrasound imaging device to adjust temperature modulating parameters. The temperature feedback device may comprise any device equivalent to a thermocouple that is inserted or implanted and is negligibly affected by viscous heating artifact. The orientation of the temperature feedback device may be controlled by a mechanical means relative to the ultrasound transducer using the needle (e.g., hypodermic needle) to guide the thermocouple into the location of the ultrasound beam. In one embodiment of the temperature feedback device shown in FIG. 7, the needle is guided at an angle relative to the ultrasound beam through an adjustable bracket that is attached or permanently a part of the transducer casing. In another embodiment of the temperature feedback device shown in FIG. 8, the needle is guided parallel and co-axial with the ultrasound beam through a small hole in the geometric center of the transducer. It is preferred that the thermocouple is encased in stainless steel to isolate the thermocouple junction from the effects of viscous heating in the fluid boundary layer between the thermocouple and the surrounding tissue.

The ultrasound heating device may further comprise an algorithm that controls temperature in a specified ROI to maximize heating within the ROI while simultaneously minimizing heating outside the ROI and minimizing the possibility of mechanical bioeffects such as cavitation within the ROI. The algorithm uses temperature feedback along with any a priori (e.g., attenuation) or a posteriori information (e.g., thermal response to past input) to control the duty-cycle, frequency, and/or intensity of the ultrasound pulses. The algorithm may be used to control only duty cycle at a constant peak-acoustic pressure and frequency to minimize the possibility of cavitation, to control the minimum peak-intensity and maximum frequency to minimize the possibility of cavitation, and/or to minimizes pulse length, peak-intensity, and/or maximizes frequency to minimize the possibility of cavitation. In one embodiment, the algorithm may comprise one or more PID control loops. In another embodiment, the algorithm may use the location of the thermocouple to account for any motion of a patient into which the thermocouple of the temperature feedback device has been inserted.

In another embodiment, the algorithm may control temperature in a specified ROI to maximize heating within the ROI while simultaneously minimizing heating outside the ROI by extrapolating or predicting 3-dimensional (3D) temperature heating patterns from one or more localized temperature measurements. The algorithm may use spatial information (e.g., anatomical information or dimension) from an ultrasound image to plan the heating treatment with or without user intervention. The algorithm may use temperature-dependent shifts in 3D ultrasound speckle pattern that quantify differential changes in temperature combined with the absolute measurements from one or more thermocouples to estimate the volumetric temperature distribution. To predict heating, the algorithm may use, e.g., a state-space model of the tissue region, a finite-element model of the tissue region to predict heating, a Kalman filter, the Pennes' bioheat transfer equation for variation in combination with a spatial model of the ROI and surrounding volume, or an approximate analytical solution to the bioheat transfer equation or variation. The Pennes' bioheat transfer equation accounts for the ability of tissue to remove heat by both passive conduction (i.e., diffusion) and perfusion of tissue by a treatment.

The ultrasound heating device may further comprise an acoustic pressure feedback device comprising a pressure sensor, e.g., piezoelectric element or elements. Preferably, the acoustic pressure feedback device is attached to and/or incorporated within a stainless steel needle. In one embodiment, the acoustic pressure feedback device provides a pressure-dependent signal that is interpretable by the ultrasound imaging device. The pressure-dependent signal may, e.g., allow the ultrasound imaging device to adjust temperature-modulating parameters through coupling to the temperature-dependent signal provided by the temperature-feedback device. The coupling may allow the output parameters of the ultrasound imaging device to be adjusted. The pressure feedback device can be used to control dose and to compensate for any patient motion. In one preferred embodiment, the pressure feedback device is incorporated in the thermocouple needle. The pressure feedback device can be used to calculate the ultrasound attenuation through the intervening medium between the transducer and a known location of the acoustic pressure sensor, determine the acoustic intensity required to heat a volume of tissue, or quickly locate the thermocouple tip with very little acoustic intensity and negligible heating. The location of the tip may be automated.

The pressure sensor of the pressure feedback device may serve as a passive cavitation detector to warn an operator or the algorithm of the presence of acoustic cavitation in the heating beam. In another embodiment, the pressure sensor, with sensitivity ranging from the subharmonic or one-half of the ultrasound frequency, may be used for heating to at least the second harmonic, or twice the ultrasound frequency may be used for heating, preferably higher, for the purpose of detecting nonlinear echoes from cavitation bubble oscillations. In another embodiment, patient and/or operator motion that causes displacement between the ultrasound beam and desired region of treatment may be estimated and compensated for by tracking the feedback from the pressure feedback device.

In another embodiment, the pressure feedback device may be used to directly control acoustic dose independently from temperature feedback.

The ultrasound heating device may further comprise a temperature sensor device. The temperature sensor device may use a fluid system comprising one or more microfluidic channels, one or more fluid pressure sensors, a controlled fluid pump, and a fluid or fluid solution with a temperature-dependent viscosity. Preferably, the temperature sensor device is non-metallic. Due to the non-metallic construction, the temperature sensor device may be suitable for use in MRI without causing artifacts. Additionally, the temperature sensor device may be constructed out of materials that are minimally attenuating and reflective to ultrasound waves. Standard instrumentation can interface the temperature sensor device to the pressure feedback device.

In one embodiment, the temperature sensor device may consist of a microfluidic channel constructed from a non-metallic material such as silicon. A fluid solution with a known temperature-dependent viscosity characteristic can be pumped through the channel with a known flow rate. The fluid pressure can be measured at the inlet to the microfluidic channel. Changes in temperature at any location along the length of the microfluidic channel change the resistance to flow within the channel. Changes in resistance can be measured as changes in pressure at the inlet to the channel. The changes in pressure can be used to determine the temperature within the channel using a pre-determined pressure versus temperature calibration for a given flow rate. The fluid system can be totally closed. The flow resistance in the fluid system can be largely determined by the microfluidic channel.

The time-dependent flow function with which the fluid is pumped into the microfluidic channel may be oscillatory (e.g., sinusoidal), so that there is no net volume of fluid pumped through the channel. For sinusoidal input function, changes in resistance within the channel can change the amplitude of the measured pressure at the inlet. Assuming that the natural frequency of the fluid system is constant (constant compliance and fluid density), the natural frequency may be used as the driving function. This can potentially give more sensitive measurement of the flow resistance and associated temperature for an underdamped fluid system. A driving frequency below or not far above the natural frequency of the fluid system may be desirable in general.

The microfluidic channel is fed by a larger diameter tube such that the microfluidic channel does not significantly load the fluid source. For example, the diameter of the inlet and outlet tubing to the microfluidic channel may be two times larger, which results in 16 (2⁴) times less flow resistance relative to the microfluidic channel under laminar flow conditions. This aspect can allow the sensitivity of the flow resistance measurement to be maintained.

In another embodiment, the temperature sensor device may consist of an array of channels along the length of a non-metallic needle that is inserted into tissue. The needle may be constructed out of a material that also has low thermal conductivity between channels, so that temperature measurements between channels are significantly independent.

The ultrasound heating device may further comprise a sub-device that interfaces a clinical imaging unit to a specialized add-on therapeutic module. Clinical scanners can be constrained in the amount of power their transmitters and power supplies can generate for safety purposes. The sub-device may connect the electrical path of a scanner and transducer to buffer the electrical pulses delivered to the transducer without significantly affecting the bi-directional propagation of electrical signals to and from the scanner. An application-specific custom array transducer may be substituted for the original transducer. The sub-device could be used in conjunction with common modes available on clinical systems including, but not limited to, pulsed-Doppler, color-Doppler, power-Doppler, M-mode, general (b-mode), and tissue harmonic. In one example application, the clinician would enter pulsed-Doppler mode, and direct the Doppler cursor (beam) to the location where heating is desired.

The sub-device may consist of a bank of bi-directional buffers that current-amplify the electrical pulses generated by a scanner. Each active channel on the scanner can have its own bi-directional buffer. The sub-device is “bi-directional”, meaning that it freely allows electrical signals to propagate in both directions for the purpose of transmission and reception on each channel.

The sub-device can be electrically isolated from a scanner and electrical ground in as much as is required to insure patient safety and meet government standards. Electrical signals propagating from the transducer back through the sub-device to a scanner may be conditioned in the sub-device for the purpose of noise filtering, amplification, attenuation, linear operations, and/or non-linear operations including, but not limited to, integration, differentiation, summation, level shifting, log-compression, or thresholding. This operation of the sub-device may also include linear and/or non-linear operations on the pulses generated by the clinical scanner.

The sub-device may also include closed-loop control of temperature using feedback from thermocouples or other means for sensing temperature. Temperature feedback may be used by the sub-device to control the intensity transmitted into the patient using a feedback algorithm to control the amplitude of the electrical signals driving the transducer. Specialized circuitry may also control the duty-cycle of the electrical signals driving the transducer.

In one embodiment, the sub-device may contain specialized logic to “learn” the input and output characteristics of channels on a clinical scanner. For example, the sub-device may use a comparator circuit to determine which channels are actively transmitting or not. In another embodiment, the sub-device may contain specialized logic that arbitrarily delays electrical signals in each channel so as to dither the resulting ultrasound beam for the purpose of spreading acoustic intensity over a larger volume.

In another embodiment, the sub-device may connect directly to an available transducer port on a clinical scanner, where the output port is either identical to the transducer port on the scanner or is a different port for a custom transducer. The sub-device may also contain specialized circuitry so that the clinical scanner is able to identify the probe connected through the sub-device.

Indicator

An indicator may be a fluorescent, luminescent, metal, magnetic, or radioactive indicator that is released into the blood stream upon activation of a temperature-sensitive carrier vehicle for the purpose of monitoring thermal treatment efficacy in tissue according to the present invention. In particular, the indicator can be used to quantify the amount of agent released from temperature-sensitive vehicles in vivo. Additionally, the indicator can be used to quantify thermal dose, blood flow in the thermally-treated region, systemic vehicle concentration, systemic concentration of released agent, and the ratio of released liposomes to intact vehicles.

In one embodiment, a temperature-sensitive liposome is loaded with a fluorescent dye, preferably a dye with peak excitation and emission wavelengths falling in the range of 650-850 nm. The encapsulated dye can be either self-quenched, or quenched by the addition of a second dye (e.g., FRET). The liposomes are injected immediately prior to heat treatment and circulate freely throughout the body. As the subject receives thermal treatment, liposomes contained in blood flowing through tissues that receive thermal treatment are released when the tissue reaches a threshold temperature that is the same temperature as the phase-transition temperature of the liposomes. The systemic concentration of the dye carried within the blood stream is monitored by an optical means in real-time during the treatment. With an estimate of the subject's blood volume and an estimate of the volume of tissue treated, the volume of blood that flows through the treated region may be estimated using the injected dye concentration (when fully released from the carrier) and the systemic dye concentration.

Multiple wavelength techniques may be employed that differentiate between encapsulated dye and released dye using, e.g., FRET between two complementary dyes. For example, circulating liposomes containing two dyes at a suitable concentration emit light at the more red-shifted dye's emission spectrum. When the dyes are released, the more blue-shifted dye is now allowed to emit with its own emission spectrum, however, the red-shifted dye's emission is much weaker. Therefore, e.g., FRET may be exploited in the proposed indicator to measure but systemic dye concentration and systemic encapsulated dye concentration at the same measurement site.

The information about the amount of dye released into the systemic circulation may be used as an indicator of the amount of drug released from liposomes that are co-injected with liposomes containing the dye. Alternatively, the dye indicators may be co-encapsulated with an agent in the same liposomes.

The indicator may be monitored invasively by drawing blood samples or through intra-vascular means of measuring concentration, such as, e.g., fiber optic probes.

The indicator may be monitored non-invasively through diagnostic imaging modalities which are sensitive to the particular indicator used, including, but not limited to, e.g., Positron emission tomography (PET), magnetic resonance imaging (MRI), nuclear magnetic resonance (NMR), single positron emission computed tomography (SPECT), computed tomography (CT), and optical imaging.

The indicator may be monitored non-invasively by an external means for accessing blood concentration, e.g., in a way similar to pulse oximetry. In one possible embodiment, the blood concentration is assessed by illuminating a finger-tip with a wavelength of light suitable for exciting a fluorescent indicator and the emitted light is filtered, detected, and quantified to give a measure of systemic concentration.

An indicator may be used that largely remains in circulation once released from a liposome. In one embodiment, the indicator may have a particularly high affinity for blood albumin or other proteins or molecules known to circulate in blood. Such an indicator would be useful when the encapsulated indicator is a fluorescent dye, encapsulated at a suitable concentration to self-quench. In this example, the released dye concentration is measured to give systemic indicator concentration.

An indicator may be used that is rapidly removed from circulation once released from a liposome. Such an indicator would be useful with the encapsulated indicator is a fluorescent dye, encapsulated at a suitable concentration to not self-quench. In this example, the encapsulated dye concentration is measured to give systemic liposome concentration.

Method

A method according to the present invention comprises stimulation of particle extravasation and uptake into tissues using a non-invasive, external means to induce inflammatory processes with spatial and temporal control, and causing release of an agent from the particles following stimulated uptake. Inflammation may be produced by irradiation with acoustic waves, electromagnetic waves, ionizing radiation, laser irradiation, microwaves.

In one embodiment, ultrasound is used to ablate small regions within and around a tumor in order to cause localized inflammation of tissue surrounding the sites of ablation. Temperature-sensitive liposomes are injected and passively accumulate in regions of inflammation due to increased vascular permeability caused by a cascade of physiologic responses directly or indirectly related to the inflammation. The contents of the liposomes are released using controlled ultrasound heating. This is one example of a method for modulating a tissue temperature and releasing an agent in a subject of the present invention.

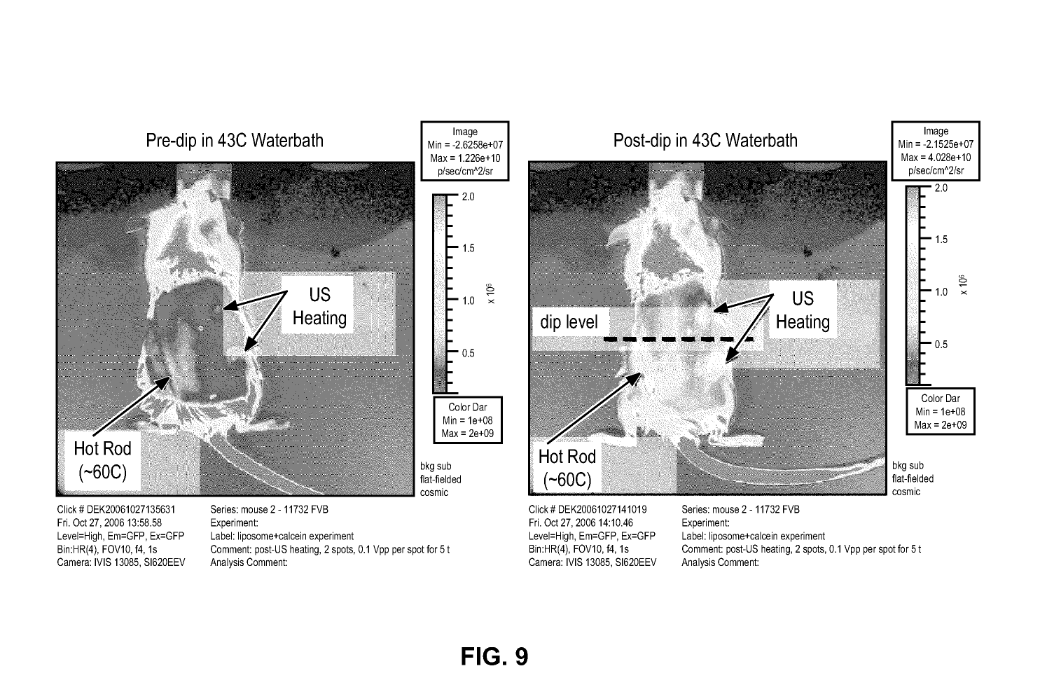

FIGS. 9(a) and 9(b) show an example of one embodiment of this method. See also Example 5 below, which demonstrates one example of a method for treating a subject using the present invention.

In one embodiment of the present invention, the ultrasound heating device utilizes spatially and temporally localized and controlled tissue ablation, significant overheating (>42° C.), cellular damage (through radiation), or mechanical damage to produce spatially controlled regions of inflammation in order to enhance the extravasation and accumulation of therapeutic agents in tissue. In another embodiment of the present invention, the ultrasound heating device includes a means for releasing the therapeutic once it has accumulated in tissue.

One aspect of the present invention addresses this issue by placing one or more acoustic pressure feedback devices on a needle in order to precisely locate the temperature feedback device within the image. The ultrasound heating device can send out acoustic pulses to rapidly find the needle within the image and spatially register the point of temperature feedback device relative to the target tissue. The needle “listens” for specific pulse patterns sent in different directions and the received radio frequency (RF) signals are processed by the ultrasound heating device to determine the location of the needle in real-time, by, e.g., using orthogonal codes unique to locations defined by a grid over the ultrasound image. Once the ultrasound heating device is “locked on” to the location of the needle, it can track changes in location of the temperature feedback device due to motion caused by patient and/or clinician. For example, a breast tumor is located using a high resolution ultrasound scanner. A thermocouple needle of a temperature feedback device is guided into the tumor, and as it's guided, the location of the temperature feedback device is displayed on the image in addition to a rendering of the needle.

Using the precise location of the temperature feedback device, a more intense ultrasound beam is simultaneously directed to the ROI, using the information from the temperature feedback device and the changes in the image to monitor the thermal dose to the desired ROI. An accurate estimate of the attenuation between the heating transducer and the known location of the acoustic pressure feedback device on the needle is made, which is used to determine the initial intensity for heating as well as derated indicies (such as the mechanical index or “MI”). The region initially is heated from the inside. As the region is heated, the beam direction and parameters are constantly adjusted to compensate for motion, e.g., from respiration. Using the thermal response acquired by the temperature feedback device along with the known beam shape, dimensions, intensity and spatial location and extent of the region, a specialized algorithm adjusts heating parameters and predicts the heating at the edge and beyond the edge of the region. Extending on this example, the needle may be instrumented with an array of thermocouple junctions and an array of acoustic sensors along its length (over several centimeters). Using a high-resolution 3-D ultrasound scanner, the region is located and the needle is directed through the center to the opposite boundary. Using the feedback from the acoustic sensors and the temperature sensors, the ultrasound beam is dithered over the entire region in 3-D to heat it uniformly according to a specialized control feedback algorithm that utilizes feedback from each temperature and acoustic sensor. Additionally, one or more independent needles may be tracked simultaneously using orthogonal codes. For example, one needle may be inserted to the center of the region and one to its outermost edge. Using the dimensions of the region and the locations of the temperature sensors, a specialized feedback control algorithm heats the region from the inside out in such a way as to achieve a uniform temperature distribution from the center to the edge of the region throughout its entire volume.

Once the temperature feedback device location within tissue is known relative to the ultrasound beam, it is possible to estimate the local thermal conductivity of the tissue immediately adjacent to the temperature feedback device. It is also possible to estimate the local blood perfusion. Both variables are useful for predicting the time course of the heating, but knowledge of the thermal properties of tissue may also be useful for identifying lesions or for monitoring the response to treatment.

Materials of the Invention

N-Succinimidyl 3-(2-pyridyldithio) propionate (SPDP), N-Succinimidyl S-Acetylthiopropionate (SATP), and N-Succinimidyl S-Acetylthioacetate (SATA) was purchased from Pierce. 1,2-dipalmitoyl-sn-Glycero-3-phosphoethanolamine (DPPE), 1, 2-distearoyl-sn-Glycero-3-phosphoethanolamine (DSPE), 1,2-dipalmitoyl-sn-Glycero-3-phosphotidylcholine (DPPC), and 1,2-diacyl-Sn-Glycero-3-Phosphoethanolamine-N-Nethoxy (Polyethylene glycol)-20001 (DSPE-PEG2000) were purchased from Avanti polar lipids. Chloroform and triethylamine were purchased from EMD. Methanol, 2-iminothiolane (2-IT), Dithiothreitol (DTT), 5,5′ dithiobis-(2-nitrobenzoic acid) (DTNB), and 2,2′-dithiodipyridine (DTP) were purchased from Sigma-Aldrich.

General

UV-Vis absorption spectra were recorded using a Varian-Cary 50 Bio spectrophotometer. Thin Layer Chromatography (TLC) was performed on plastic backed 20×20 cm silica gel 60 sheets. Particle sizing was performed with a NiComp 380 ZLS. ESI Mass spectra measurements were obtained on a Thermo Finnigan Mass Spectrometer. A 10 liter stock of 10× Phosphate buffered saline (PBS) was prepared by dissolving 800 g NaCl, 20 g KCl, 144 g Na₂HPO₄ and 24 g KH₂PO₄ in 8 L of distilled water, and topping up to 10 L. PBS can also be formulated to contain calcium or magnesium.

Methods and Compounds of the Invention

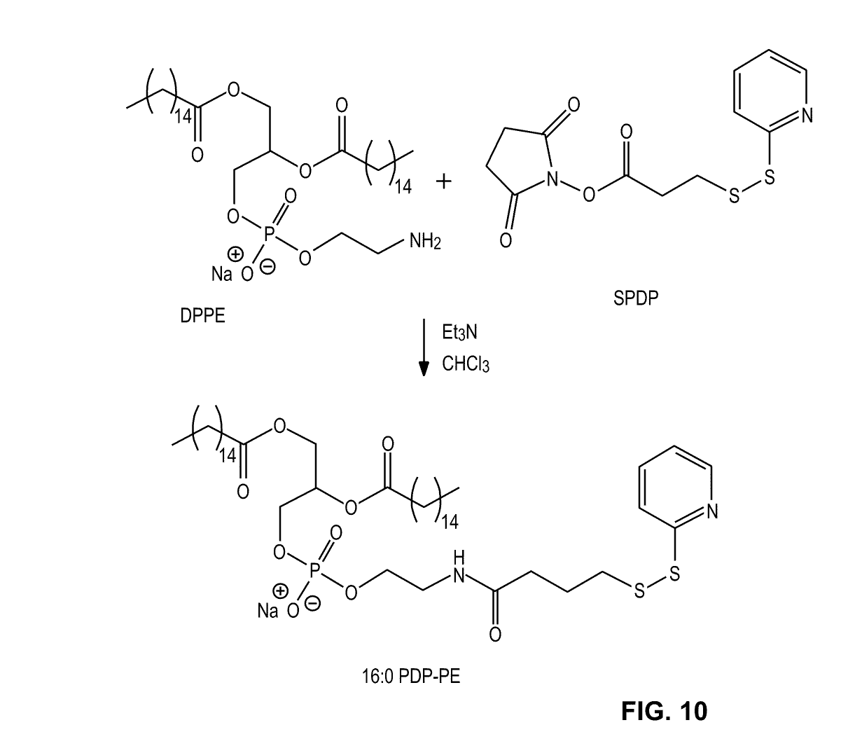

FIG. 10 shows Synthesis of N-[3-(2-Pyridyldithio) propionyl]-1,2-dipalmitoyl-sn-glycero-3-phosphoethanolamine (16:0 PDP-PE).

Initially, 25 mg (36 mmol) of DPPE in 2 ml chloroform was added to 17 μL (0.12 mmol) triethylamine (TEA) in a vial with a stir-bar. 17.1 mg (54.7 mmol) of SPDP was added to the mixture while stirring under argon at room temperature. The solution was stirred for 4 hours, then TLC was performed with a mobile phase of chloroform, methanol, and water in 65/25/4 v/v/v ratio. The fluorescamine test for amines was negative for the crude mixture, confirming conjugation. See Udenfriend, S., Stein, S., Bohlen, P., Dairman, W., Leimgruber, W., and Weigele, M. (1972) Fluorescamine: A Reagent for Assay of Amino Acids, Peptides, Proteins, and Primary Amines in the Picomole Range, Science, 178, 871-872.

The crude mixture was added to 2 ml chloroform and 4 ml 18 MΩ water. The solution was centrifuged at 2000 rpm for 5 min, afterwards the aqueous layer was extracted. 4 ml of water was added to the organic layer and the extraction process was repeated for a total of four times.

TLC was performed on the organic and aqueous phases and the product was visualized using a phosphorous detection reagent (Ellingson, J. S. & William E. M. Lands (1968) Phospholipid Reactivation of Plasmalogen Metabolism. Lipids. 111-120). No phosphorous was detected in the aqueous layer. The organic layer containing product was dried under vacuum. The product was stored under chloroform. The concentration was determined using UV-Vis spectrophotometry at 343 nm (ε=8.08×10³ cm⁻¹ M⁻¹) (Carlsson, J. (1978) Protein Thiolation and Reversible Protein-Protein Conjugation. Biochem. J. 173, 723-737.). Product yield was 3.49 mg (38%). ES-MS m/z 934 ([M+Na⁺]), calcd 934 for C₄₅H₈₀N₂Na₂O₉PS₂.

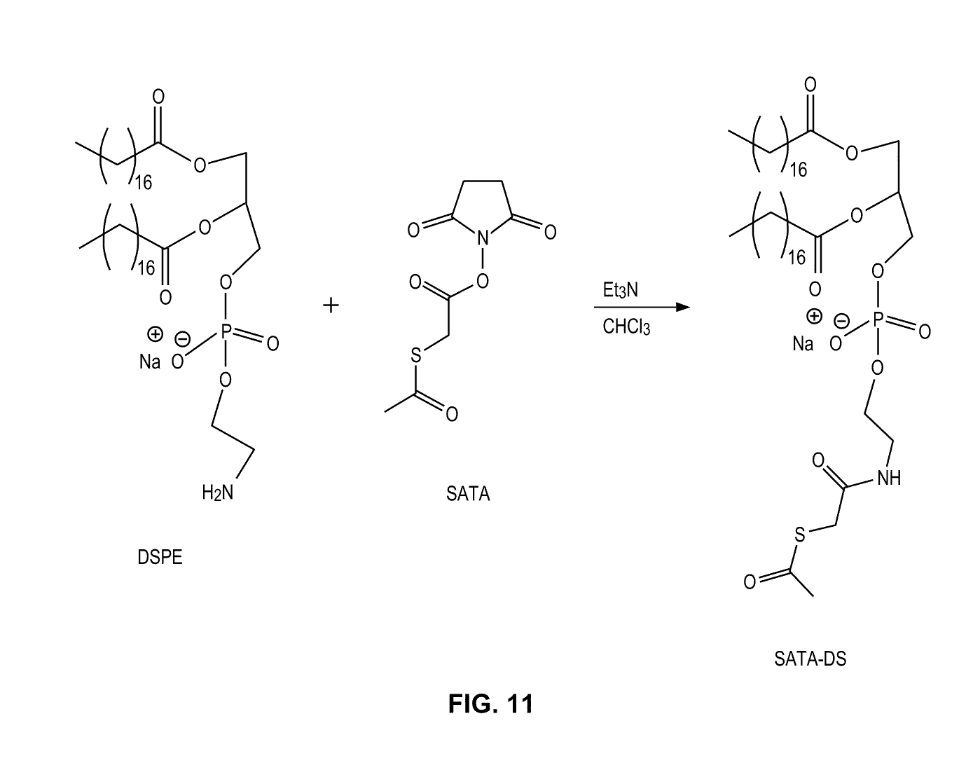

FIG. 11 shows synthesis of SATA-DSPE.

25 mg (33.4 mmol) of DSPE in 2 mL chloroform was added to 17 μL (0.12 mmol) triethylamine in a vial with a stir-bar. 11.6 mg (50.2 mmol) of SATA was added to the mixture while stiffing under argon at room temperature. The solution was stirred overnight, then TLC was performed with a mobile phase of chloroform, methanol, and water in 65/25/4 v/v/v ratio. The fluorescamine test for amines was negative for the crude mixture.

The crude was placed in a test tube with 2 mL of chloroform, a drop of methanol, and 4 mL of 18 MΩ water. The solution was centrifuged at 2000 rpm for 5 minutes, after which the aqueous phase was decanted and 4 mL of water was added. This process was repeated a total of four times. The aqueous phase fractions were added together, dried under reduced pressure and resuspended in chloroform.

TLC was performed on the fractions and the product was visualized using a phosphorous spray. The fraction containing product were combined and dried under vacuum. The product was stored under chloroform. The concentration was determined by first deprotecting the sulfhydryl group with hydroxylamine from a procedure according to Pierce, then performing the DTNB test (Ellman, G. L (1959). Tissue Sulfhydryl Groups. Arch. Biochem. Biophys. 82, 70-77.; Riddles, P. W., Blakeley, R. L. Zerner, B. (1983) Reassessment of Ellman's Reagent. Methods Enzymol. 91. 49-60.). The yield was 2.14 mg, 7.25% of the desired phospholipid. ES-MS m/z: 909.6 ([M+Na⁺]), calculated is 909 for C₄₅H₈₅NNa₂O₁₀PS.

Synthesis of SATA-DPPE

25 mg (36.1 mmol) of DPPE in 4 mL chloroform was added to 17 μL (0.12 mmol) triethylamine in a vial with a stir-bar. 12 mg (51.9 mmol) of SATA was added to the mixture while stiffing under argon at room temperature. The solution was stirred for four hours, then TLC was performed with a mobile phase of chloroform, methanol, and water in 65/25/4 v/v/v ratio. The fluorescamine test for amines was negative for the crude mixture.

The crude was placed in a test tube with 4 mL of chloroform, and 4 mL of 18 MO water. The solution was centrifuged at 2500 rpm for 5 minutes, after which the aqueous phase was decanted and 4 mL of water was added. This process was repeated a total of four times. The aqueous phase fractions were added together, dried under reduced pressure and resuspended in chloroform.

TLC was performed on the fractions and the product was visualized using a phosphorous spray. Fractions containing product were combined and dried under vacuum. The product was stored under chloroform. The concentration was determined by first deprotecting the sulfhydryl group with a procedure according to Pierce then performing the DTNB, Ellman's Reagent, test. The yield was 0.8 mg (2.67%) of the desired phospholipid. ES-MS m/z: 853.7 ([M+Na⁺]), calculated is 853.08 for C₄₁H₇₇NNa₂O₁₀PS.

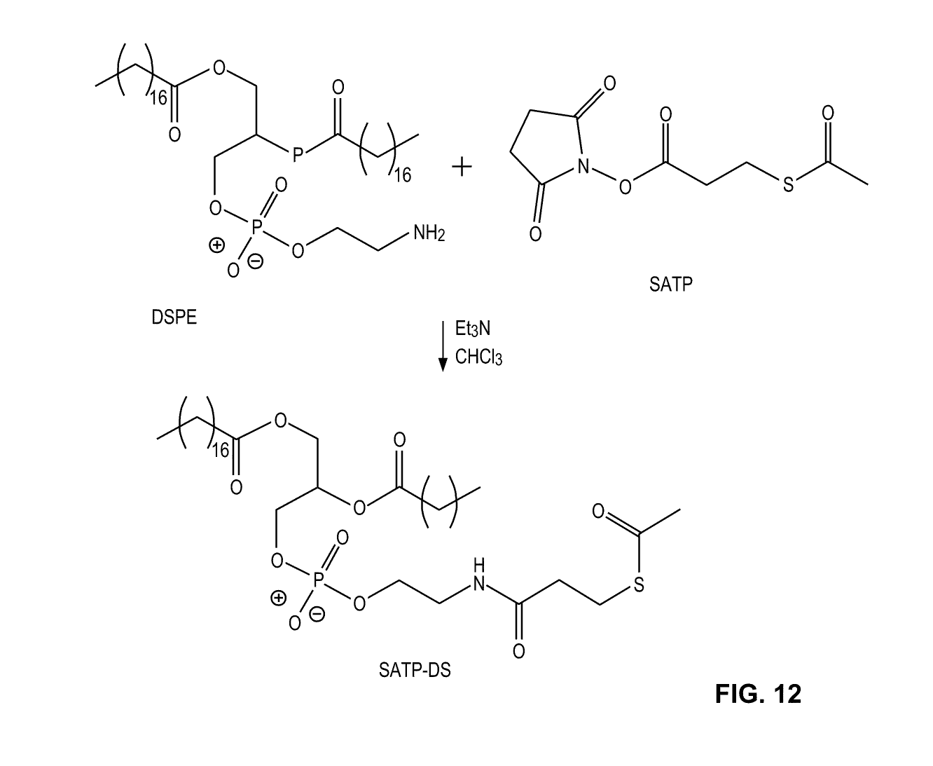

FIG. 12 shows synthesis of SATP-DSPE.

25 mg (33.4 mmol) of DSPE in 4 mL chloroform was added to 17 μL (0.12 mmol) triethylamine in a vial with a stir-bar. 11.6 mg (50.2 mmol) of SATA in 2 mL methanol and added to the mixture while stirring under argon at room temperature. The solution was stirred for 4 hours, then TLC was performed with a mobile phase of chloroform, methanol, and water in 65/25/4 v/v/v ratio. The fluorescamine test for amines was negative for the crude mixture.

The mixture was purified by flash silica gel chromatography with an Analogix RS-12 silica column using a chloroform 69%/methanol 27%/water 4% solvent system. TLC was performed on the fractions and the product was visualized using a phosphorous spray. Fractions containing product were combined and dried under vacuum. The product was stored under chloroform. The concentration was determined by first deprotecting the sulfhydryl group with a procedure according to Pierce then performing the DTNB test. The yield was 1.4 mg, 6.4% of the desired phospholipid. ES-MS m/z: 923.5 ([M+Na⁺]), calculated is 923.21 for C₄₆H₈₇NNa₂O₁₀PS.

Pharmaceutical Compositions of the Invention

Methods for treatment of diseases also are within the scope of the present invention. Said methods of the invention include administering a therapeutically effective amount of a composition of the present invention. The composition of the invention can be formulated in pharmaceutical compositions. These compositions can comprise, in addition to one or more of the vehicles, a pharmaceutically acceptable excipient, carrier, buffer, stabilizer, or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, or intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol, or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

Administration of the composition is preferably in a “therapeutically effective amount” or “prophylactically effective amount,” (as the case can be, although prophylaxis can be considered therapy), this being sufficient to show benefit to the individual. The actual amount administered, and rate and time-course of administration, will depend on the nature and severity of the disease being treated. Prescription of treatment, e.g., decisions on dosage, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed.), 1980.

A composition can be administered alone or in combination with other treatments, either simultaneously or sequentially dependent upon the condition to be treated.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### Example 1

### Vehicle Formation and Testing with DPPC, DSPE-PEG2000, DSPE, and PDP-PE

DPPC (13.7 mmol), DSPE-PEG2000 (0.806 mmol), DSPE (1.61 mmol), and PDP-PE (1.61 mmol) were mixed together and dried overnight under nitrogen. The mixture was placed in 300 μL of borate buffer, which consisted of 0.112 M boric acid/NaOH pH 10.1, 5 mM EDTA, and 0.15 M NaCl. The solution was sonicated for 4 minutes at 51° C. then centrifuged for a minute to remove the lipids from the side of the vial. The extruder was heated to 90° C. after the lipids were added to the syringe. The lipids were extruded 25 times through a 100 nm nucleopore membrane. After extrusion, the particles were sized using a NiComp 380 ZIS particle sizer.

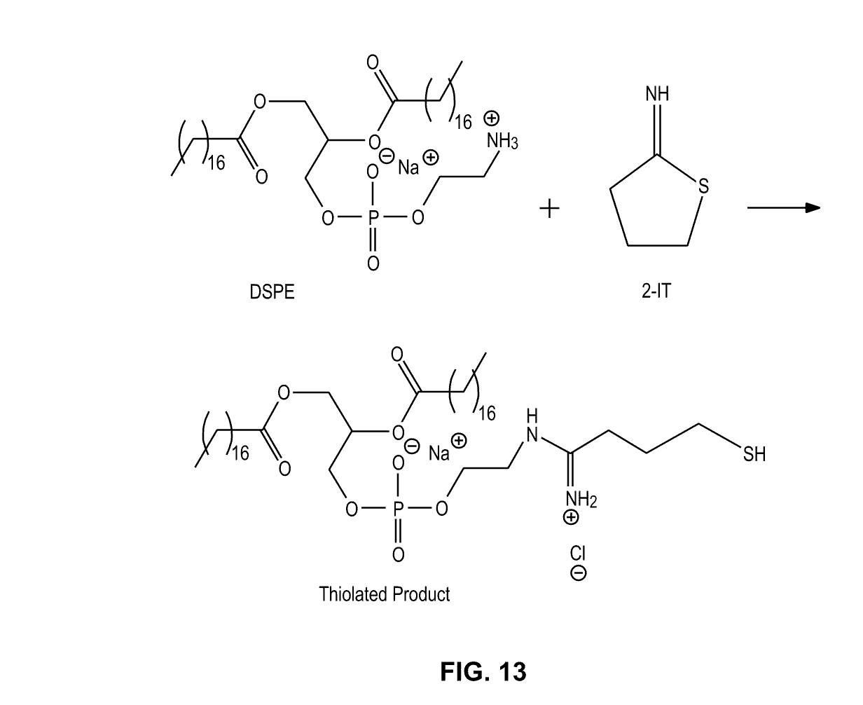

FIG. 13 shows reaction of liposomes with 2-IT.

A similar procedure was previously described in Lasch, J. Niedermann, G. Bogdanov, A. A, Torchilin, V. P. (1987) Thiolation of Preformed Liposomes with Iminothiolane. FEBS. 214, 1, 13-16.

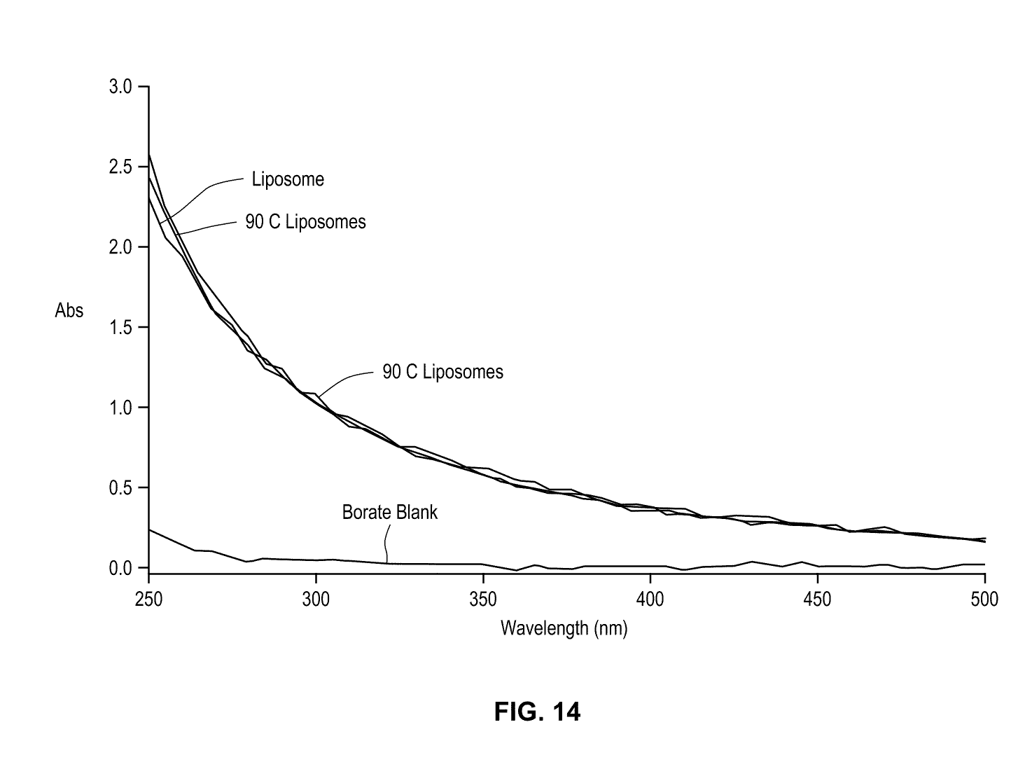

To the formed 100 nm liposomes, a solution of 110 μL of 0.5 M 2-IT was added and gently shaken for 40 minutes. UV-Vis spectra were taken before addition of 2-IT, directly after addition, and periodically during the reaction. The mixture was then added to a G-75 sephadex column with a mobile phase of borate buffer. The fractions containing the liposomes were collected and then UV-Vis spectra, as shown in FIG. 14, were collected of the liposomes before and after heating to ˜90° C. for 10 minutes

### Example 2

### Vehicle Formation and Testing with DPPC, DSPE-PEG2000, SATA-DSPE, and PDP-PE

9.49 mg (12.9 mmol) of DPPC, 2.42 mg (0.86 mmol) of DSPE-PEG2000, 1.53 mg (1.72 mmol) of SATA-DSPE, and 1.57 mg (1.72 mmol) of PDP-PE were added together and dried overnight under vacuum. 500 μL of phosphate buffered saline (PBS) was added to the lipid mixture and heated to 55° C. The mixture was sonicated for three seconds to break up large micelles, and then extruded through a 100 nm filter 31 times while the extruder was heated to 80° C. Liposomes were sized, then diluted with an additional 200 μL of PBS and re-extruded. The liposomes were sized again, and then added to a G-75 Sephadex column. The fractions were sized to ensure that they contained the desired 100 nm liposomes and then collected.

Tests using DPPC, DSPE-PEG2000, SATA-DSPE, and PDP-PE.

To deprotect the thiol group on the liposomes 100 μL of 0.5 M hydroxylamine with 25 mM EDTA pH 7.3 in PBS buffer were added to 50 μL of PBS buffer containing the liposomes. The mixture was gently mixed over an hour. The procedure for deprotection was taken from Pierce.

50 μL of liposomes formed by the second method were diluted into 650 μL of PBS. UV-Vis spectra were taken of PBS as a blank, the liposomes in PBS, and hydroxylamine solution in PBS all at room temperature.

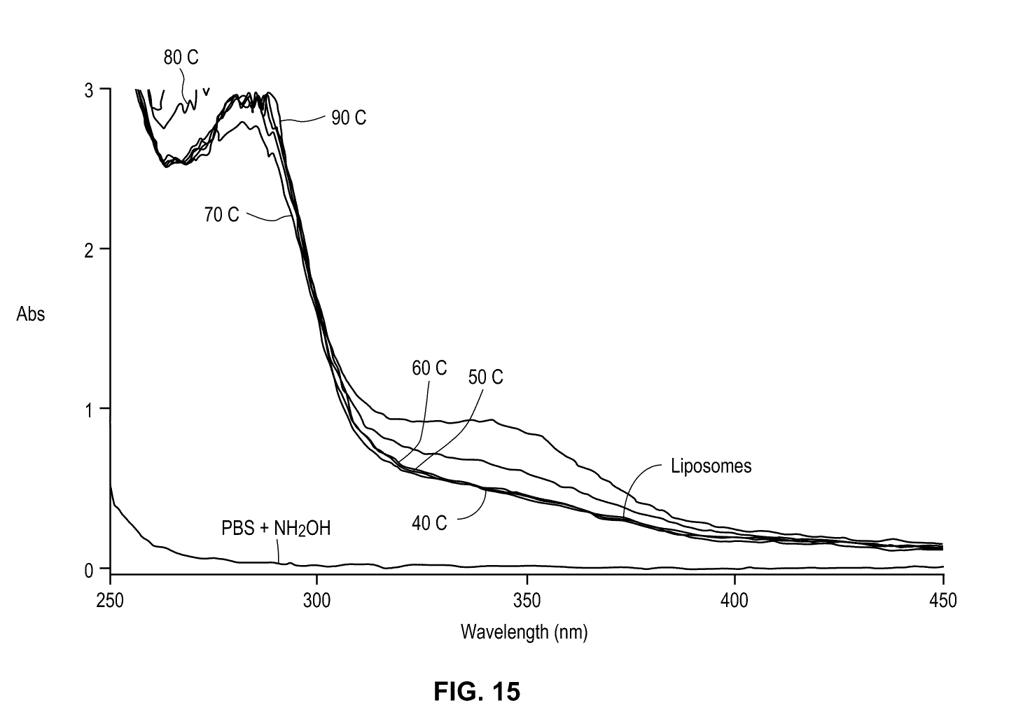

The liposomes were heated to 40 to 90° C. at 10 degree intervals for 10 minutes each, upon cooling to room temperature UV-Vis spectra was taken; the spectra are shown in FIG. 15.

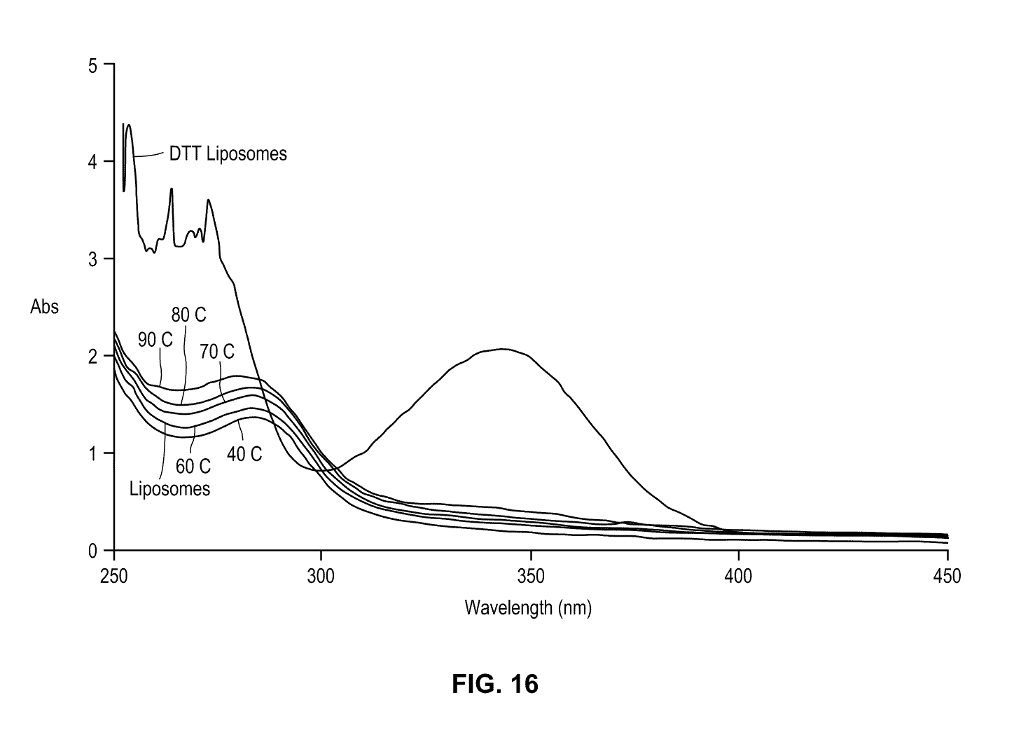

As a negative control, the same procedure was performed on the same concentration of liposomes and PBS without the hydroxylamine solution. After heating the negative control to 90° C. DTT, which serves to cleave any disulfide bonds present, was added to visualize the amount of PDP-PE that is present in the liposomes, as shown in FIG. 16.

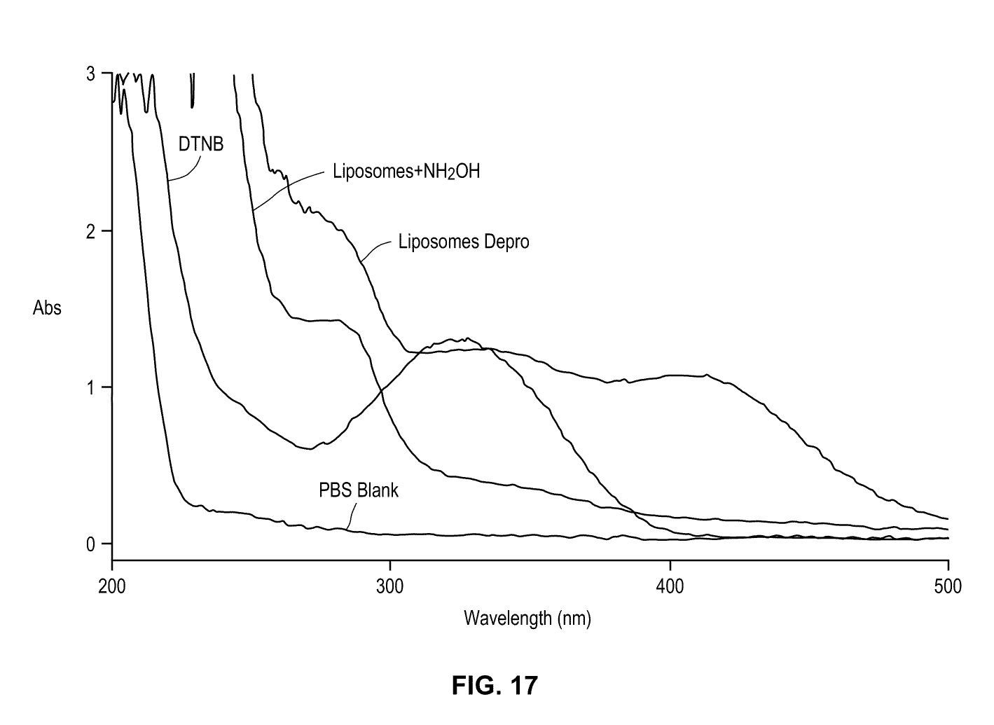

In order to determine the amount of SATA-DSPE present in the liposomes, a similar concentration of liposomes as the proof of concept experiment was added to the deprotection hydroxylamine solution overnight. The solution was then treated with DTNB, UV-Vis spectra were taken at each step, the spectra are shown FIG. 17.

UV-Vis Analysis of Disulfide Bond Stability to Heat and Hydroxylamine

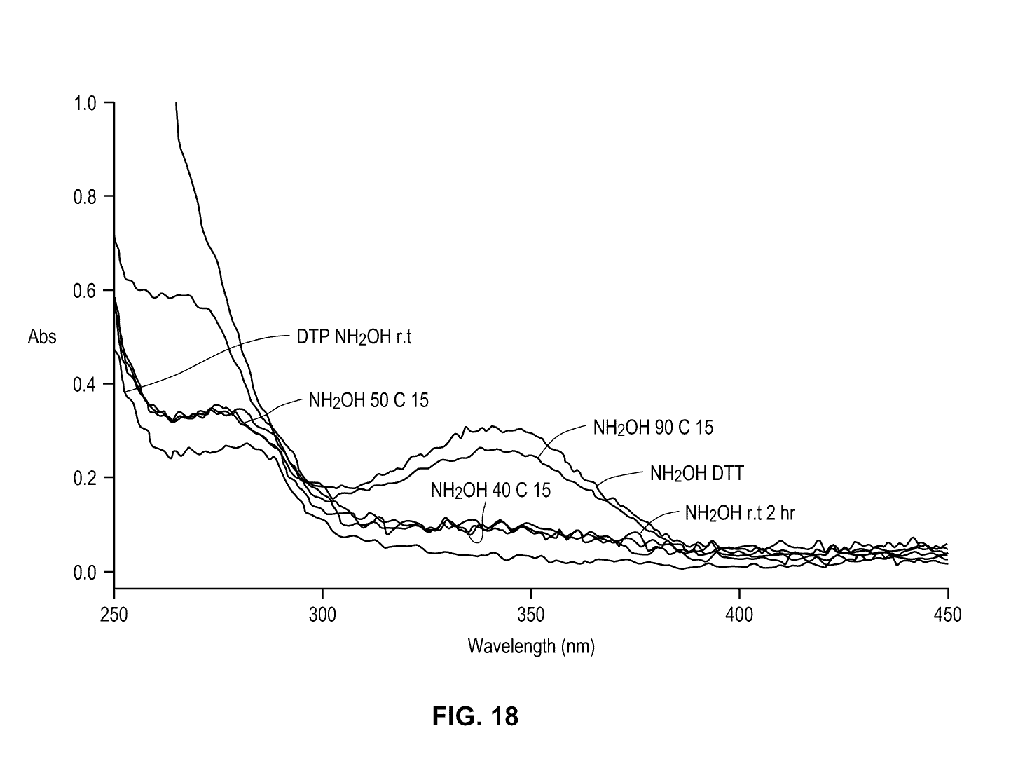

FIG. 18 shows UV-Vis spectra that was taken of 3 μL of 2.16 mM 2,2′ dithiodipyridine and 50 μL of 0.5 M hydroxylamine and 25 mM EDTA at pH 7.5 in 147 μL PBS buffer at room temperature, 40, 50, 60, 70, 80, and 90° C. The solution was heated for 15 minutes at each temperature and allowed to cool to room temp before the spectra were taken. ˜1 mg of DTT was added to the solution to cleave the remaining disulfide bonds.

UV-Vis Analysis of Disulfide Bond Stability to Heat

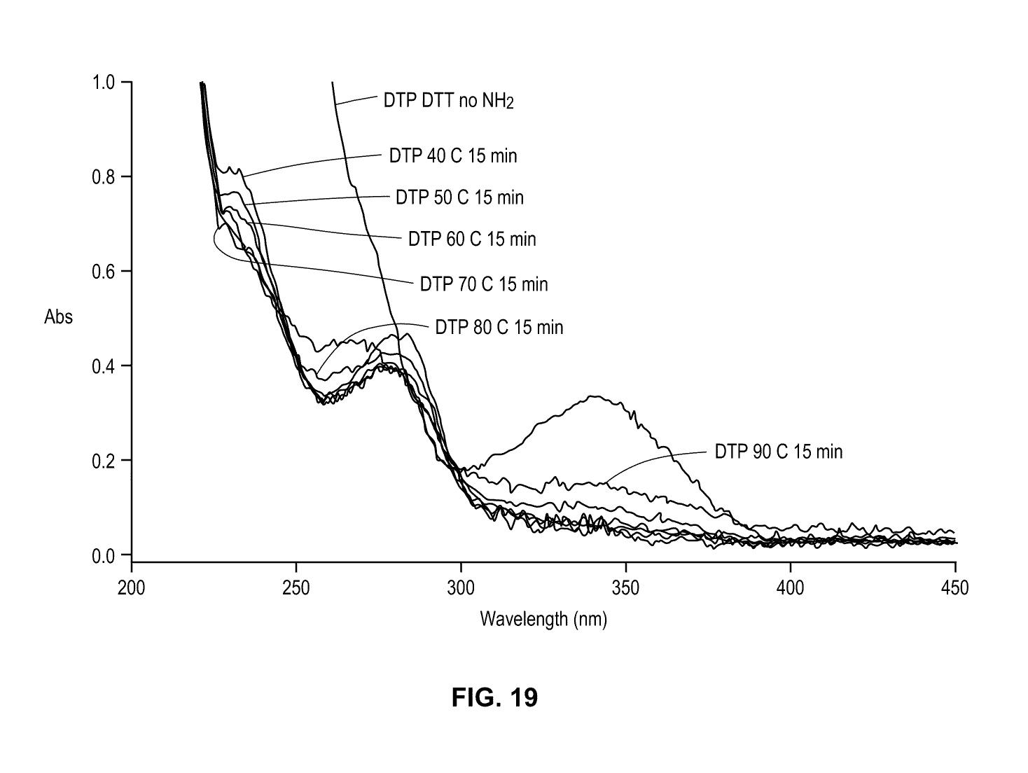

FIG. 19 shows UV-Vis spectra that was taken of a 3 μL of 2.16 mM 2,2′ dithiodipyridine in 197 μL PBS buffer at room temperature, 40, 50, 60, 70, 80, and 90° C. The solution was heated for 15 minutes at each temperature and allowed to cool to room temp before the spectra were taken. ˜1 mg of DTT was added to the solution to cleave the remaining disulfide bonds.

This method allowed for the addition of a protected thiol before the formation of the liposomes, which can be deprotected without causing the liposome to melt or bombarding the surface of the liposome with the reducing agent. In the first trial, detection of the chromophore can be seen when the temperature was around 80° C. as shown in FIG. 15. Comparing the experiment to a similar concentration of liposomes that was treated with DTT, it can be seen that not all of the chromophore was reduced. By sonicating the control but not the proof of concept samples, the liposomes can be broken apart, exposing more of the chromophore.

Heating of 2,2′ dithiodipyridine with and without the hydroxylamine solution present shows that reduction is occurring as shown in FIG. 18-19. FIG. 16 shows that PDP-PE does not seem to be reduced with only heat. From this data, one of ordinary skill in the art would understand that the chromophore was reduced by the intended mechanism.

### Example 3

### Vehicle Formation and Testing with DPPC, DSPE-PEG2000, DPPE, SATP-DSPE, and PDP-PE

5.58 mg (7.6 mmol) of DPPC, 2.57 mg (0.9 mmol) of DSPE-PEG2000, 0.3 mg (0.44 mmol) of DPPE, 1.18 mg (1.31 mmol) of SATP-DSPE, and 1.19 mg (1.31 mmol) of PDP-PE were added together and dried overnight under vacuum. 300 μL of PBS was added to the lipid mixture and heated to 55° C. The mixture was sonicated for three seconds to break up large micelles, and then extruded through a 100 nm filter while the extruder was heated to about 90° C. The liposomes were sized (data not shown), and then added to a G-75 Sephadex column. The fractions were sized (data not shown) to ensure that they contained the desired 100 nm liposomes and then pooled together.

Tests Using DPPC, DSPE-PEG2000, DPPE, SATP-DSPE, and PDP-PE

To deprotect the thiol group on the liposomes 10 μL of 0.5 M hydroxylamine with 25 mM EDTA pH 7.5 in PBS buffer were added to 90 μL of PBS buffer containing 50 μL of liposome stock. The mixture was gently mixed over two hours. UV-Vis spectra were taken of PBS as a blank, the liposomes in PBS, and hydroxylamine solution in PBS all at room temperature.

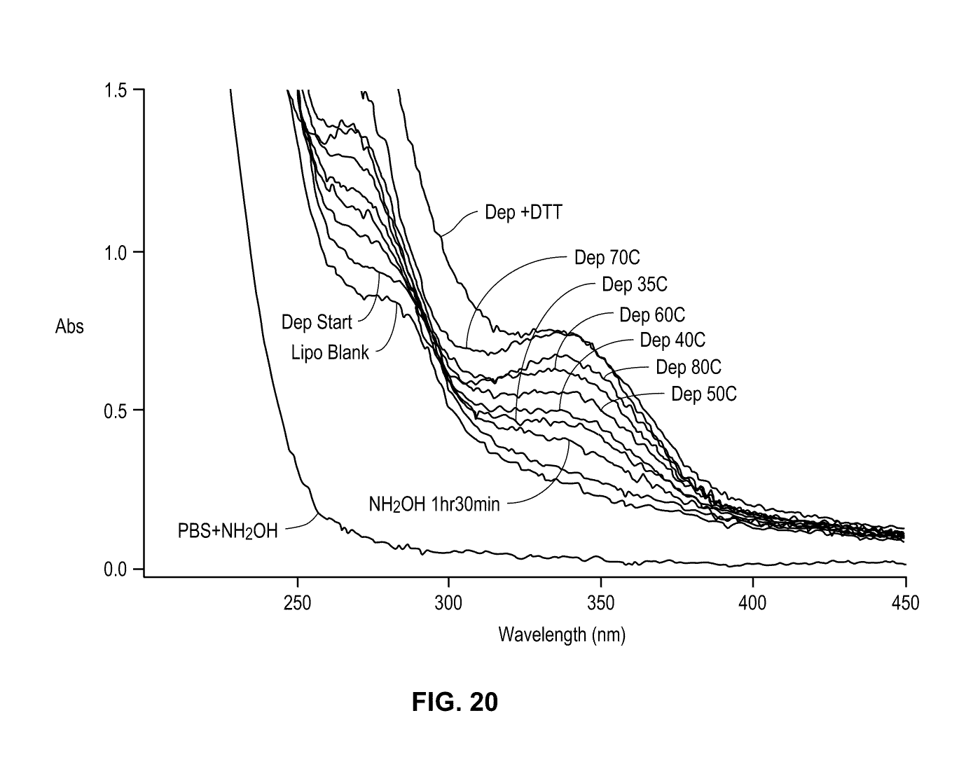

The liposomes were heated to 35, 40, 50, 60, 70, and 90° C. for 10 minutes each, upon cooling to room temperature UV-Vis spectra was taken at each interval as shown in FIG. 20. The liposomes were then treated with 3 μL of 2.5M DTT to gauge the amount of total chromophore present on the liposomes.

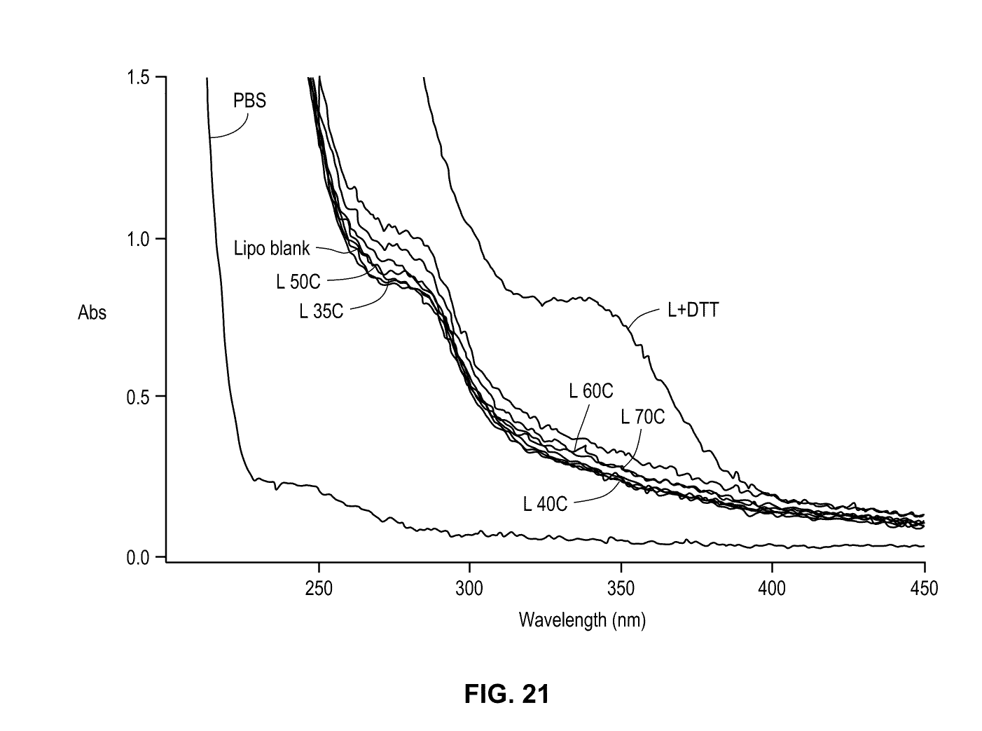

As a control, the same procedure was performed on 50 μL of liposomes stock in 100 μL PBS buffer without the hydroxylamine solution. After heating the negative control to 80° C., 3 μL of 2.5M DTT, which serves to cleave any disulfide bonds present, was added to visualize the amount of PDP-PE that is present in the liposomes as shown in FIG. 21.

Effects of Heat and Hydroxylamine Solution on Liposomes

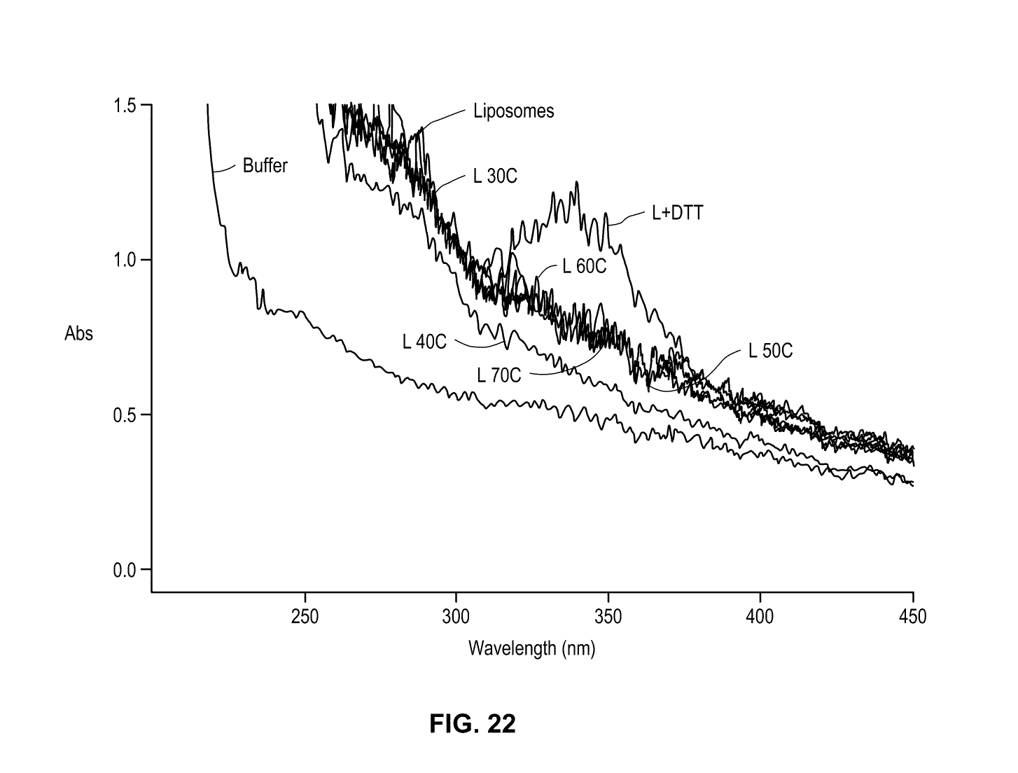

To determine if the heating or the hydroxylamine solution had an appreciable effect on the results, liposomes were made using 1.05 mg (1.15 μmoles) of PDP-PE and 12.7 mg (17.3 μmoles) of DPPC using the same procedure described earlier. 180 μL of PBS buffer was added to 20 μL of liposomes stock. The sample was then heated to 30, 40, 50, 60, and 70° C. for 15 minutes each; at each interval UV-Vis spectra were taken of the sample. After heating the sample to 70° C., the sample was treated with 5 μL of freshly prepared 0.3 M DTT solution to calculate the amount of chromophore present in the sample as shown in FIG. 22.

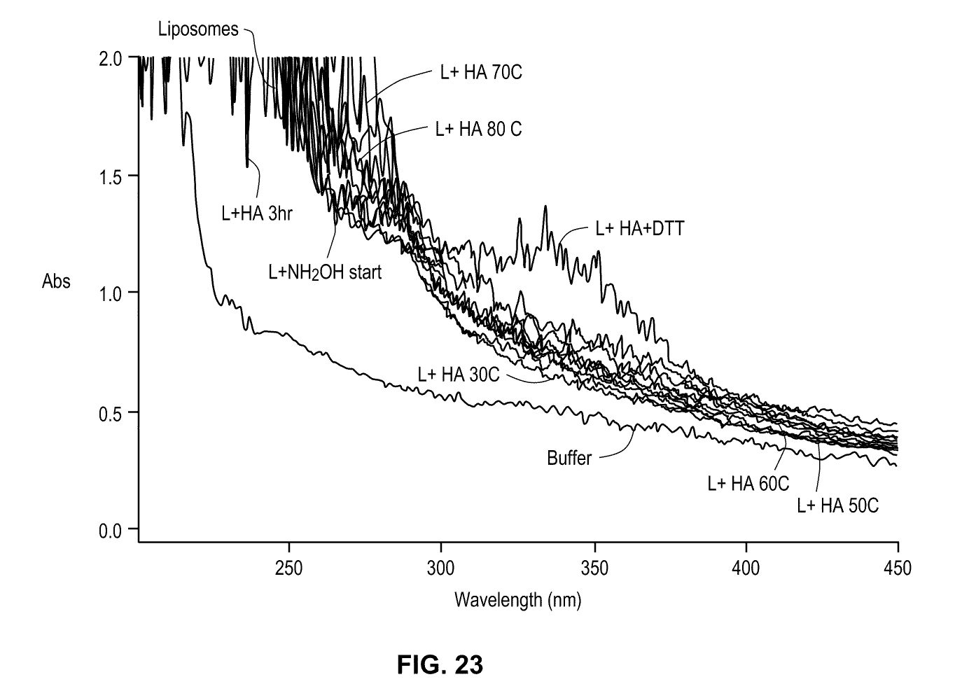

This procedure was repeated with the difference being that the liposomes were added to 160 μL PBS buffer and 20 μL of freshly prepared 0.5M hydroxylamine 25 mM EDTA pH 7.2. UV-Vis spectra were taken of the sample before and directly after addition of the hydroxylamine solution as shown in FIG. 23. The sample was incubated in a closed Eppendorf tube for 2 hours before continuing the experiment.

While some modifications of the original proof of concept experiment were made, the overall goal was observed, which was: temperature-mediated release of the chromophore.

This method allowed for the addition of a protected thiol before the formation of the liposomes, which can be deprotected without causing the liposome to melt or bombarding the surface of the liposome with a disulfide reactive agent.

From the heating experiment with the liposome containing only PDP-PE it can be commented that neither heat nor hydroxylamine has a significant effect on the release of the chromophore. These data indicate that the intended mechanism of release occurred in the proof of concept.

The mechanism for temperature controlled release of a compound was tested with success, with controls to eliminate possibly unwanted mechanisms of release of the chromophore. This method offers a mechanism of controlled release of a compound by utilizing a physical property of liposomes; the lack of convoluted chemical methods of release demonstrates the usefulness of this method.

### Example 4

### Heating with an Ultrasound Heating Device

The ultrasound heating device has been tested in vitro using a tissue mimicking phantom consisting of 3% agarose, 1.5% silicon carbide particles, and 95.5% water (all by weight). The phantom material is submerged in a water bath with an ultrasound transducer. The water bath couples the ultrasound energy into the tissue phantom. The thermocouple is inserted into the tissue phantom and located by moving the transducer beam with a micrometer stage and looking for a spike or sudden change in the temperature. The location of the focus of the transducer gives the highest steady-state temperature within the phantom. The high sensitivity of the thermocouple allows for a relatively small acoustic intensity to find the thermocouple location. Typically, a temperature rise of no more than 1 degree Centigrade (C) is required. Once the thermocouple is located, the control loop is switched on and controls the temperature to a user-defined set-point temperature. The modified PID loop is able to maintain the temperature at the thermocouple tip to within 0.1 degree C. indefinitely within the tissue-mimicking material, as shown in FIG. 6.

### Example 5

### Method for Heating with an Ultrasound Heating Device to Release Agents

FIGS. 9(a) and 9(b) show an example of one embodiment of this method. An anesthetized mouse was shaved and chemically depilated to remove all hair on its back, two lesions were created in the skin using one second-duration pulses of high intensity ultrasound (right flank) Immediately after ultrasound heating, 100 uL of a solution containing liposomes encapsulating self-quenched calcein was injected through the tail vein. The liposomes were allowed to circulate for approximately 10 minutes. The animal was imaged in a Xenogen IVIS 100 imaging system; the fluorescent image is shown in FIG. 9(a). The animal was then dipped in 43 degrees C. water for 30 seconds up to a level submerging only one of the two spots. The resulting fluorescent image is shown in FIG. 9(b). It is apparent that the submerged spot gained a significant degree of fluorescence intensity due to the release of encapsulated calcein. It is also apparent that release of the dye is concentrated in a ring around the site of ultrasound ablation as is consistent with earlier experiments performed by Kruse et al. involving similar treatments to transgenic mice containing heat-shock-protein-70-promoted luciferase expression.

### Example 6

### Release of Chromophores by Flip-Flop Mechanism

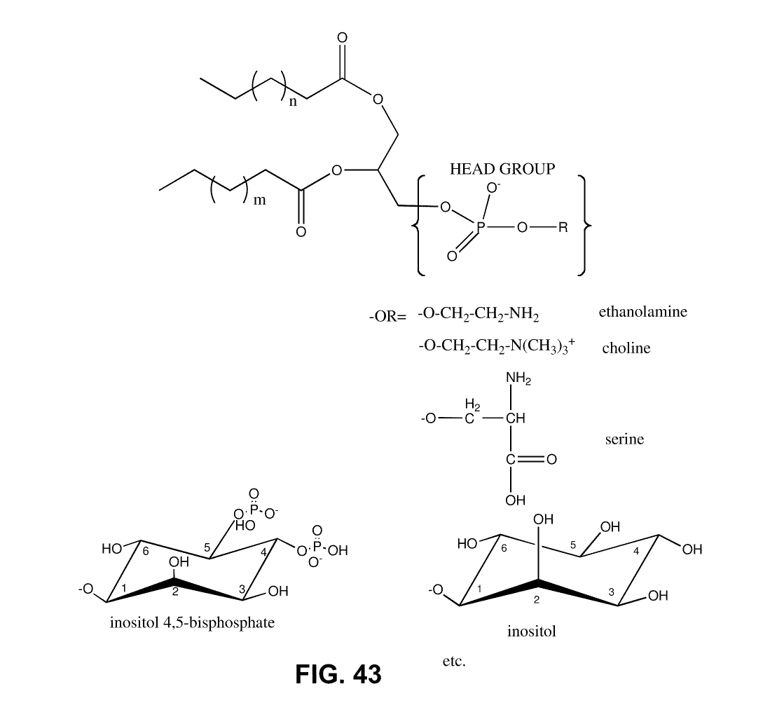

The flip-flop method utilizes the bilayer movement of lipids to control payload release. The flip-flop method can use an asymmetric bilayer distribution of thiols on one side and disulfides on the other. Below the melting temperature of the liposomes the flip-flop rate is low, but, as the temperature approaches and reached the melting temperature, the rate is greatly increased allowing interaction of the head-groups of the lipids. This study examined the effects of altering the liposomes composition, such as the acyl chain length and charge of the probe lipid as well as the bulk lipid, on the rate of flip-flop. Several liposomal formulations were tested to minimize the chromophore release at 37° C., while maximizing release at 42° C. It was observed that the counter-ion to the synthesized probe lipids also affected the flip-flop rate. A comparison of triethylammonium versus sodium analogues of liposome flip-flop release is shown. A chromophore was chosen to serve as a model compound for the diagnostic or therapeutic agents that can be used in the practice of this invention, as release of the chromophore is easily monitored. Substitution of diagnostic or therapeutic agents for the chromophore described in this example is well within the level of ordinary skill, and will not change the principles by which this invention operates. As one of ordinary skill will recognize, a diagnostic or therapeutic agent used in the present invention will require the presence of a functional sulfur-containing group capable of participating in a thiol-disulfide exchange reaction (or other functionally equivalent or similar reactions) as described in this specification. In addition, one of ordinary skill will recognize that changing the head group, e.g. from ethanolamine to serine, can be used to modify the rate of drug release from a vehicle of the invention. Head groups can include natural head groups and/or artificial head groups. In general, other head groups can include any alcohol esterified to the phosphate or any alcohol that comprises a reactive group, e g, amine, thiol, another alcohol, carboxyl, phosphate, azide, aminooxy, hydrazine, hydrazide, ketone, aldehyde, ester, thioester, or alkyne. Other examples of head groups are shown in FIG. 43 and can also be found on the Avanti Polar Lipids, Inc. web-site on Jul. 21, 2009.

A liposome can be a spherical bilayer comprised of phospholipids that surround a core of fluid. Liposomes have been researched extensively for drug delivery to tumors. Liposomes are able to accumulate in the vascular tissue of the tumor, due to the enhanced permability and retention effect(1). The encapsulated contents of liposomes will slowly diffuse through the membrane bilayer, thus exposing the surrounding cellular membrane to the contents. Exploitation of this property allows for a smaller dose of toxic chemotherapeutics, while at the same time, not sacrificing efficacy of the drug. The method of delivery has been the passive diffusion of drugs through the liposome membrane to the surrounding cellular environment. This method of drug delivery is seen in several FDA approved liposomal drug formulations such as Doxil®, DaunoXomes®.

Development of temperature sensitive liposomes, or TSL, has been shown to improve drug release by utilizing the phase transition temperature, also known as the melting temperature, of the liposomes(2). The mechanism of release is due to pores are formed in the membrane at the melting temperature, allowing leakage of the drug to occur. A common lipid 1,2-dipalmitoyl-sn-glycero-3-phosphocholine, DPPC, has a melting temperature of 41° C., allowing mild hyperthermia to enhance drug release.

Liposomes, antibodies, and polymers have taken advantage of intracellular reducing agents (i.e., enzymatic) to cleave a disulfide containing pro-drug(3,4).

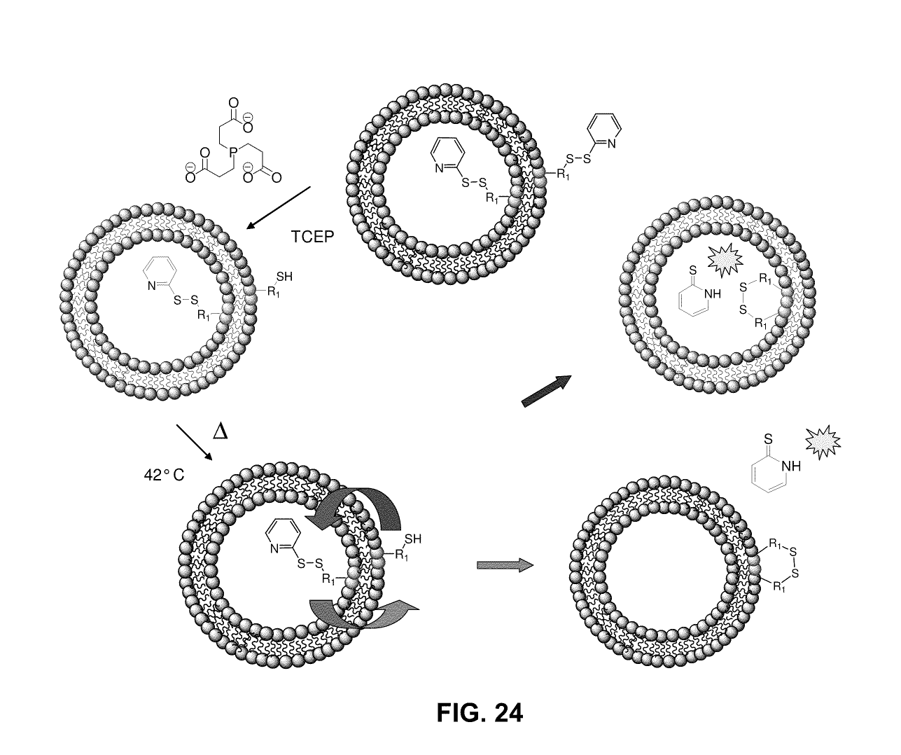

The flip-flop, a physical property of lipids in liposomes, is a process by which a lipid will exchange between the layers of the liposome. This process, while not fully understood(5), undergoes a rate increase at the phase transition temperature(6) and has been shown to assist in the partitioning of anthracyclines thru liposomes(7). The process is energetically unfavorable since it involves moving a polar, sometimes charged, head-group through the non-polar acyl chains of lipids. By using selective reducing agents on preformed liposomes containing a lipid with a disulfide(8,9), an asymmetric distribution of thiols and disulfides can be produced on the bilayers of a liposome (FIG. 24). The rate of the reaction will be dictated by the flip-flop rate, which can be modulated with temperature. This approach can be used to conjugate and controllably release small molecules either on the inside or outside of the liposome. Since the payload is covalently bound to the liposome, activation and release of the drugs mainly occur at the areas which are heated.

The method of drug release (FIG. 24) relies on a thiol-disulfide reaction on the head groups of phospholipids. The reaction releases pyridine-2-thione, a chromophore that can be quantified by UV-Vis spectroscopy. This model allows quantification of release of a payload, which will be then be applied to actual drug delivery from liposomes.

Materials and Methods

General

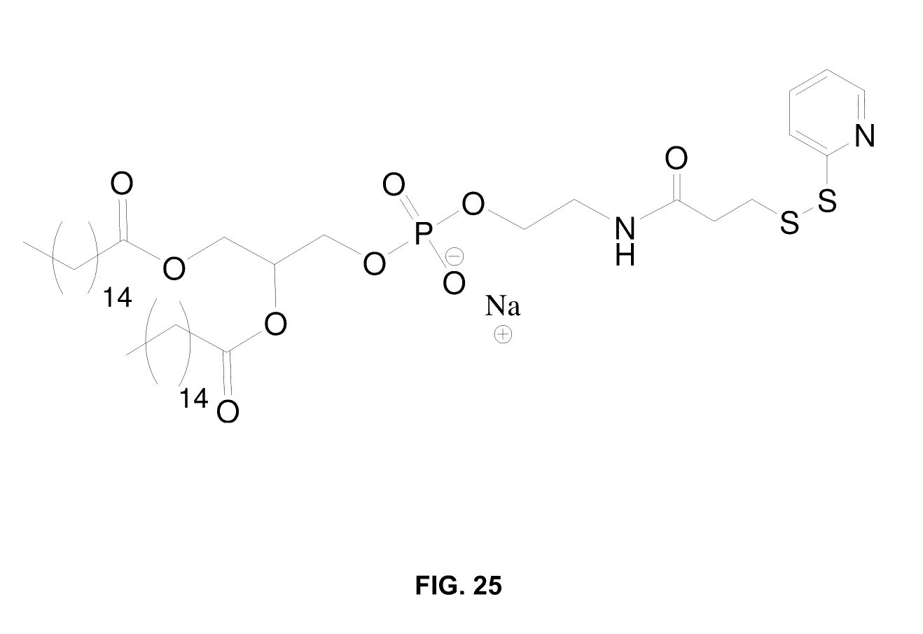

All lipids and a mini-extruder were purchased from Avanti Polar Lipids (Alabaster, Ala.). N-Succinimidyl 3-(2-pyridyldithio) propionate (SPDP) was purchased from Molecular Biosciences. TCEP was purchased from Pierce. DTT was purchased from Promega (Madison, Wis.). All other chemicals were purchased from commercial vendors. The probe lipids seen in FIGS. 25-27 were synthesized previously.

General

Instrumentation

Dynamic Light Scattering. Liposome particle sizing was performed with a NICOMP 380 ZLS (Santa Barbra, Calif.).

Ultraviolet-Visible Spectroscopy. UV-Vis spectra were collected from a Varian-Cary bio 50 using a 1-cm-path-length temperature jacketed cell connected to a circulating water bath. A quartz cuvette was used for all measurements.

Liposome Formation and Characterization

LUV were formed by mixing lipids together in formulations stated in the tables, and using nitrogen gas to remove the organic solvents. The liposomes were lyophilized overnight to remove any remaining solvents. 1 mL of PBS buffer pH 7.4 was added to the lipid film and the suspension was heated to 60° C. for 1 hour with gentle shaking and vortexing. The suspension was extruded through a 100 nm polycarbonate filter mounted on a heated mini-extruder. Particle size of the liposomes was performed by dynamic light scattering to ensure liposomes are approximately 100 nm. The liposomes were stored at 4° C. and discarded after two weeks.

Abbreviations for Example 6

16:0 PDP-PE—1,2-dipalmitoyl-sn-glycero-3-phosphoethanolamine-N-[3-(2-pyridyldithio) propionate

18:0 PDP-PE—1,2-distearoyl-sn-glycero-3-phosphoethanolamine-N-[3-(2-pyridyldithio) propionate.

CaCl₂—Calcium chloride

CHCl₃—Chloroform

DMPE—1,2-dimyristoyl-sn-glycero-3-phosphoethanolamine

DPPC—1,2-dipalmitoyl-sn-glycero-3-phosphocholine

DPPE—1,2-dipalmitoyl-sn-glycero-3-phosphoethaolamine

DSPC—1,2-distearoyl-sn-glycero-3-phosphocholine

DSPE—1,2-distearoyl-sn-glycero-3-phosphoethanolamine

DSPE-PEG2000—1,2-distearoyl-sn-glycero-3-phosphoethanolamine-N-[methoxy (polyethylene glycol)-20001 (ammonium salt)

DTT—Dithiothreitol

DTNB—5,5′-Dithio-bis(2-nitrobenzoic acid)

DTP—2IT-DPPE

ESI-MS—Electrospray ionization mass spectrometry

HPLC—High Performance Liquid Chromatography

KCl—Potassium chloride

KH₂PO₄—Potassium phosphate monobasic

MeOH—Methanol

MgCl₂—Magnesium chloride

NaCl—Sodium chloride

Na₂CO₃—Sodium carbonate

NaHCO₃ Sodium bicarbonate

Na₂HPO₄—Sodium phosphate dibasic

NaOAC—Sodium acetate

NMR—Nuclear Magnetic Resonance

PBS—Phosphate Buffered Saline, 0.9 mM CaCl₂ 0.49 mM MgCl₂, 2.7 mM KCl, 138 mM NaCl, 1.5 mM KH₂PO₄, 8.06 mM NaHPO₄ pH 7.4

SPDP—N-Succinimidyl 3-(2-pyridyldithio) propionate

LUV—Large Unimellar Vesicles

TCEP—tris (2-carboxyethyl) phosphine

Tₘ—Phase transition temperature of liposomes or lipids—melting temperature

TNB—2-nitro-5-thiobenzoate—Reduced form of DTNB

TLC—Thin layer chromatography

TSL—Temperature Sensitive Liposome

UV-Vis—Ultraviolet-Visible

UV-Vis measurements of Flip-flop release

Experiments were performed as follows. UV-Vis spectra were collected at each point, or addition of components. 100 uL of 1 mM chromophore lipid-containing liposomes in PBS pH 7.4 were placed in a quartz UV-Vis cuvette. Liposomes were then purified away from excess TCEP by a G-75 Sephadex column. Fractions from the column were tested for the presence of liposomes by UV-Vis by measuring the light scattering. The column fractions were tested for the presence of TCEP by performing a DTNB(13,14) spot test for reducing agents. The freshly-reduced liposomes were placed in a quartz cuvette, sealed with a teflon cap and parafilm to reduce evaporation. A full UV-Vis spectrum was collected, which was labeled time zero and was used as the baseline. Baseline subtracted spectra from 800-300 nm were collected periodically, 15 min or 30 min per scan. A circulating water bath was set at the specified temperatures. When the temperature of the water reached the set temperature, UV-Vis spectra were collected. The temperature was held constant over the course of the experiment. After 1 day, the liposomes were treated directly with approximately 3 mg DTT, 40 mM final concentration. It was observed that an additional treatment of DTT was not necessary. A full spectrum scan was collected 15 minutes after addition of DTT. The absorbance at 343 nm after treatment with DTT was used as the 100% release mark. The results were plotted as percent release versus time. Percent release=(A₃₄₃ (At time X)−A₃₄₃ (time zero)/(A₃₄₃ (After DTT)—A₃₄₃ (time zero)). The absorbance at 800 nm was subtracted from the absorbance at 343 nm if significant baseline drift occurred.

Results

The release of Chromophore by Flip-Flop Mediated thiol-disulfide exchange

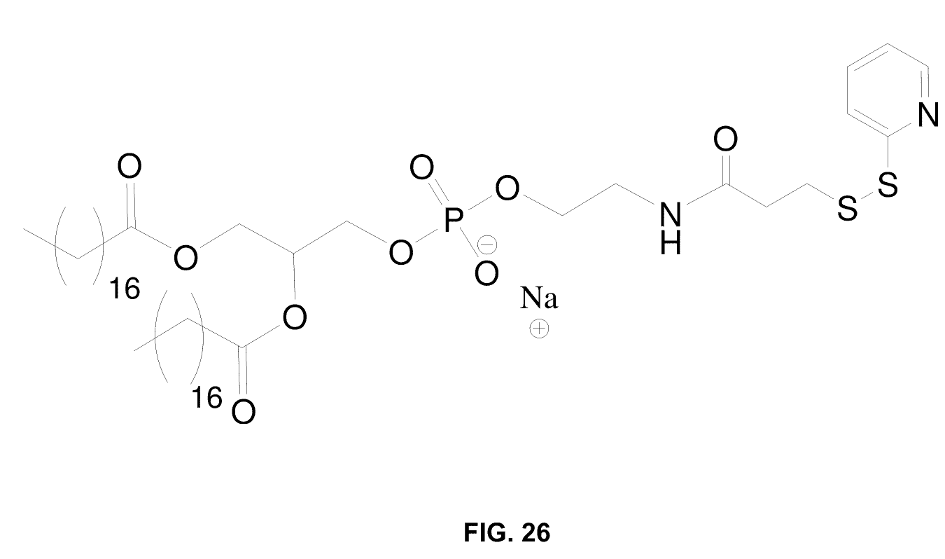

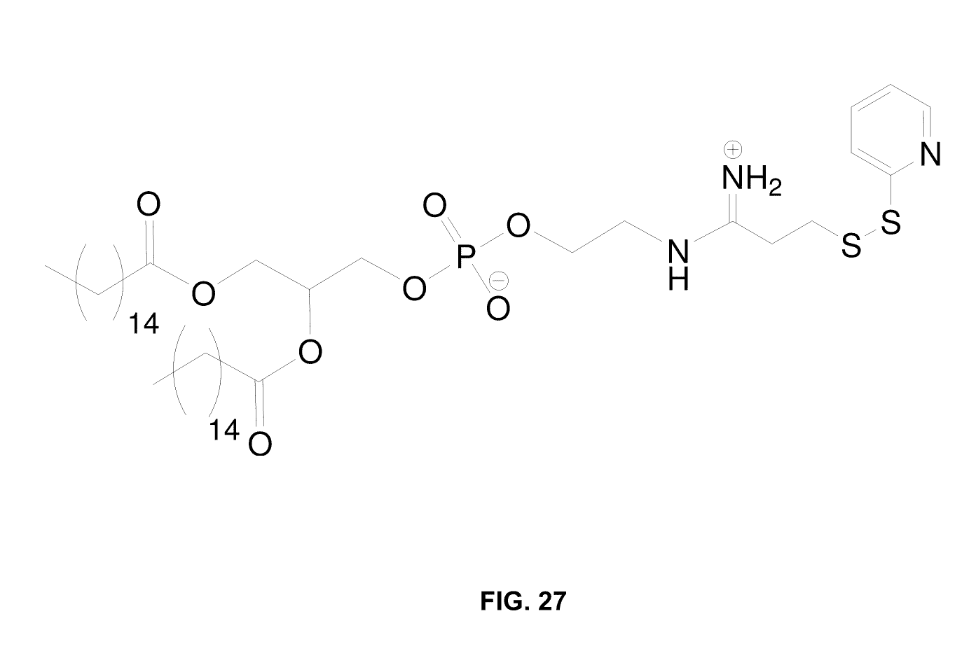

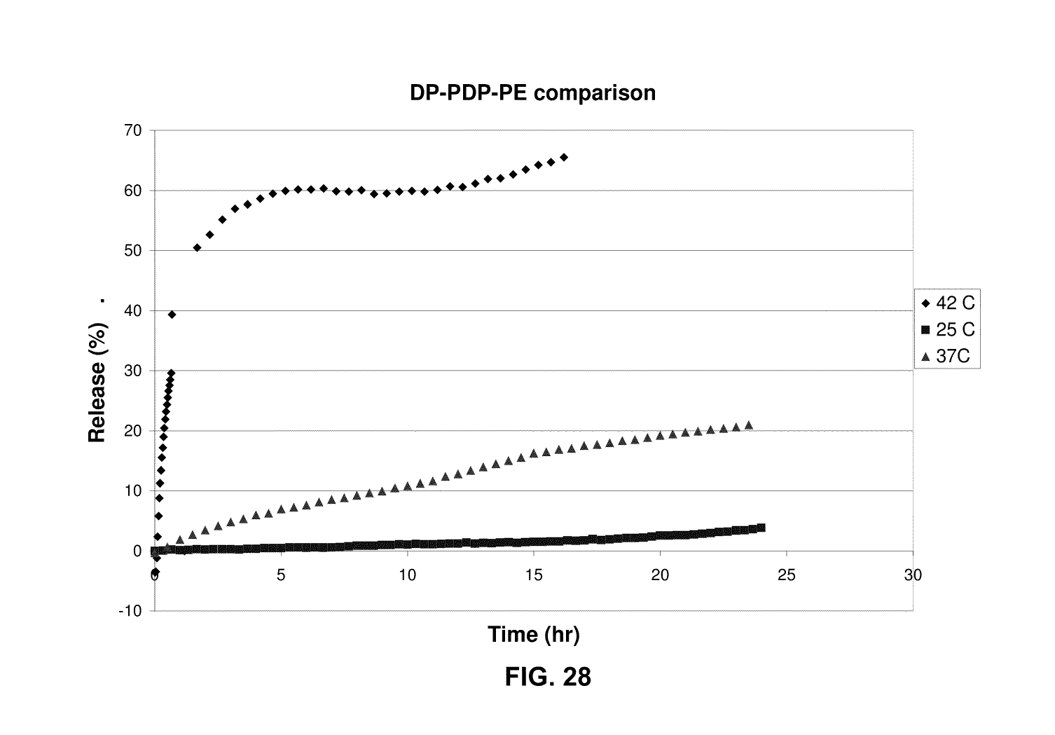

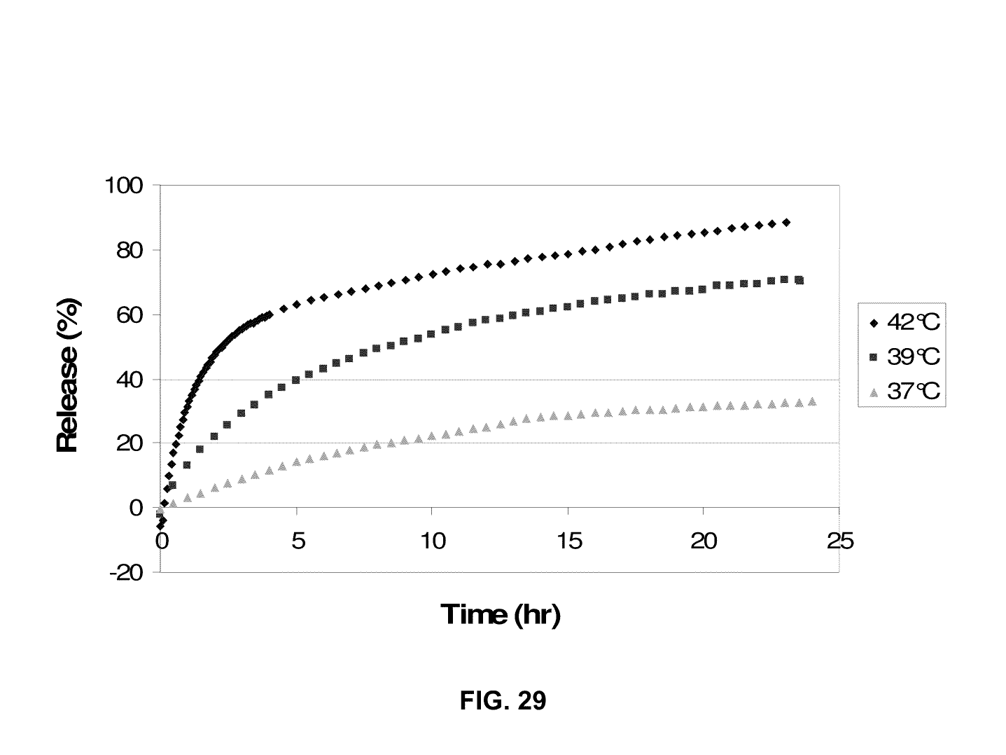

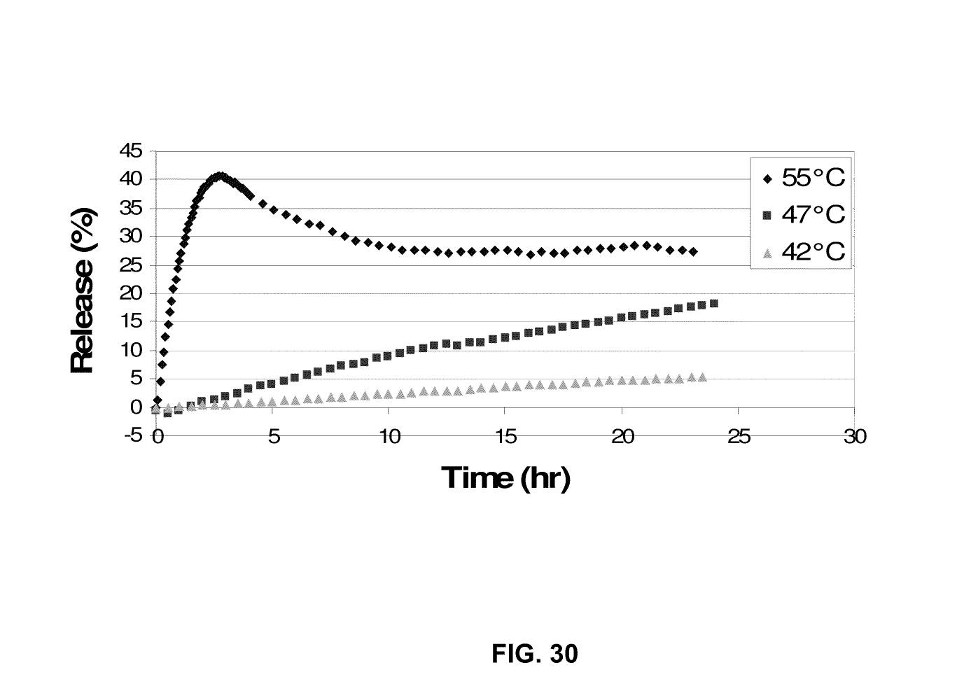

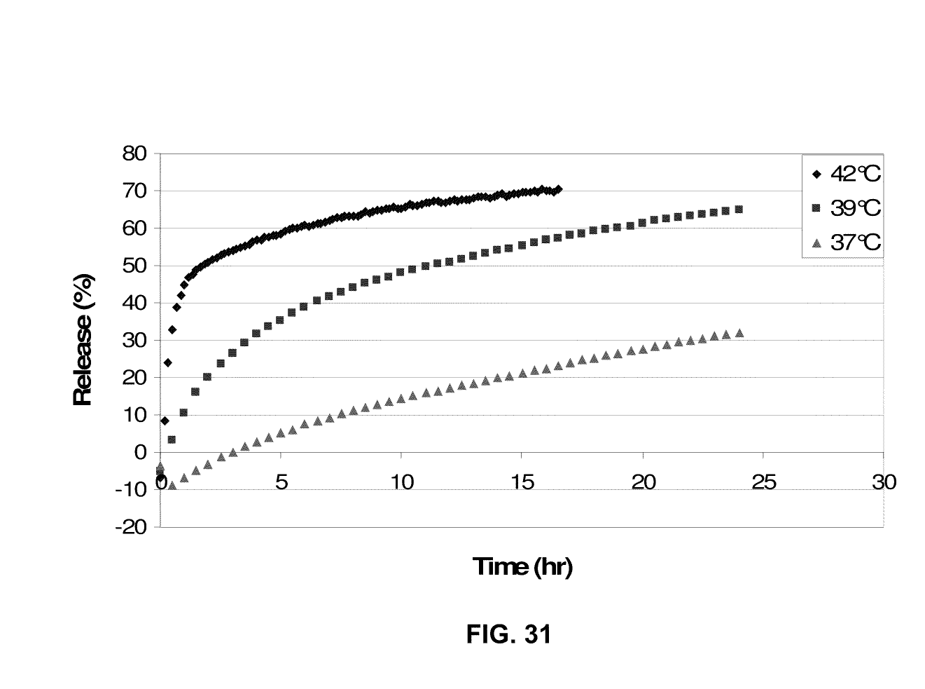

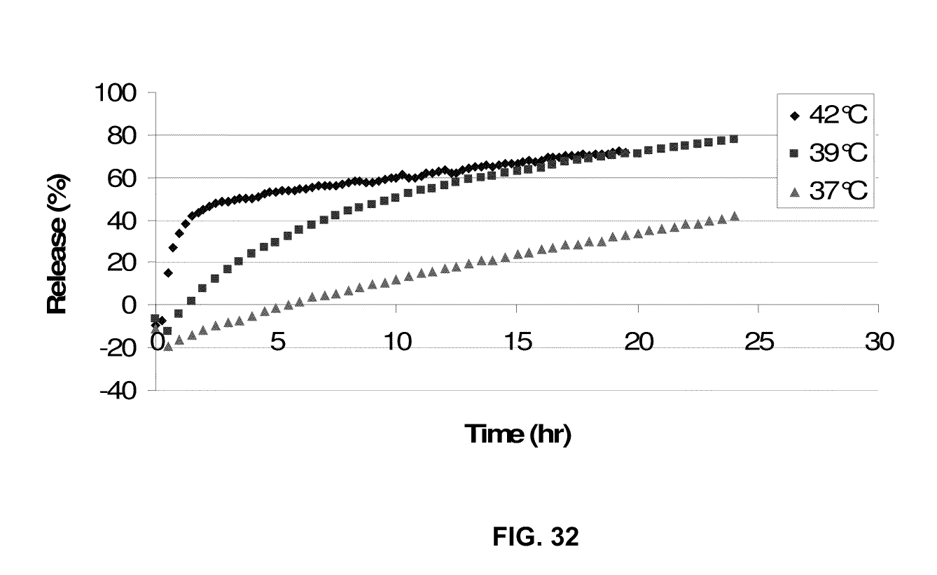

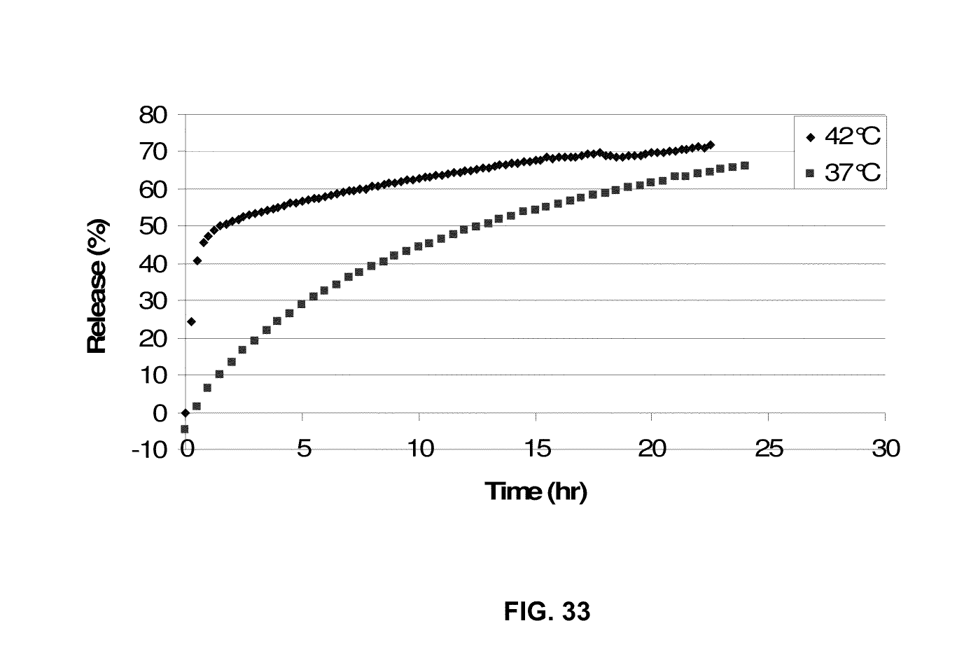

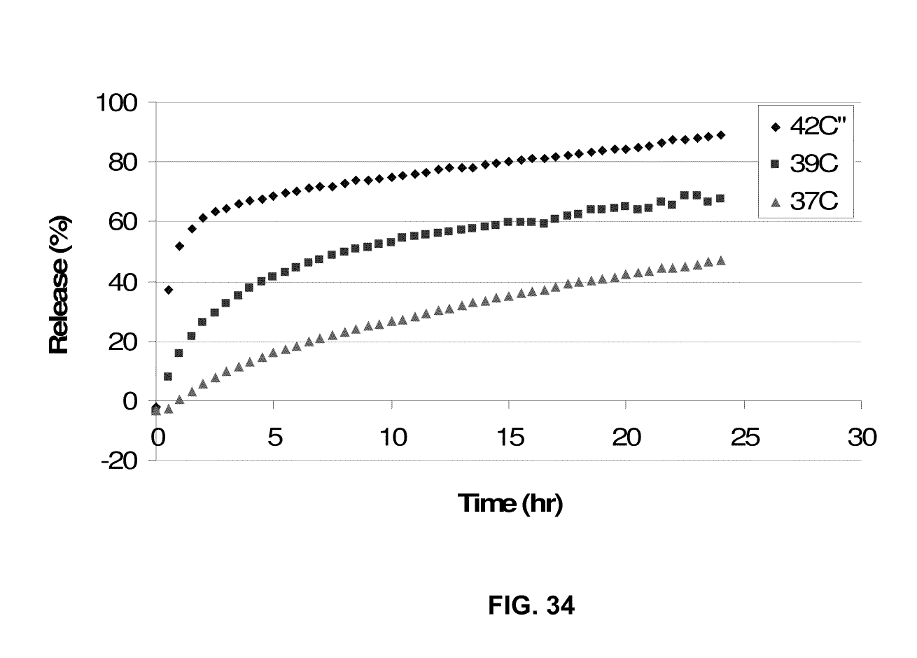

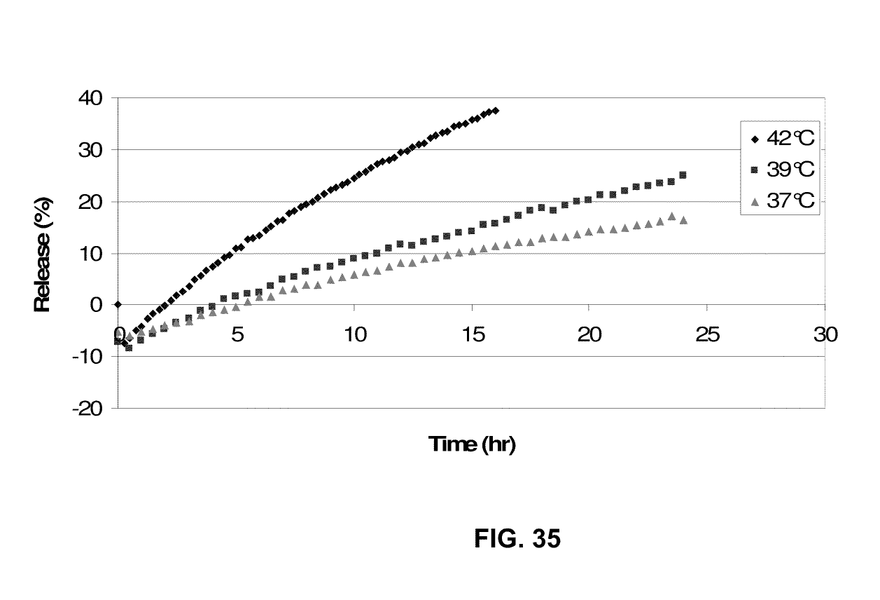

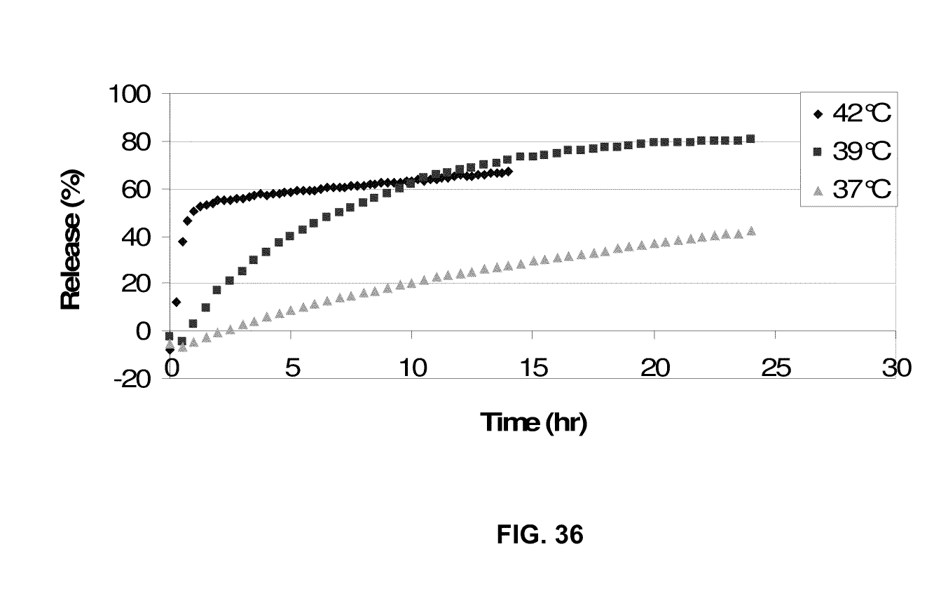

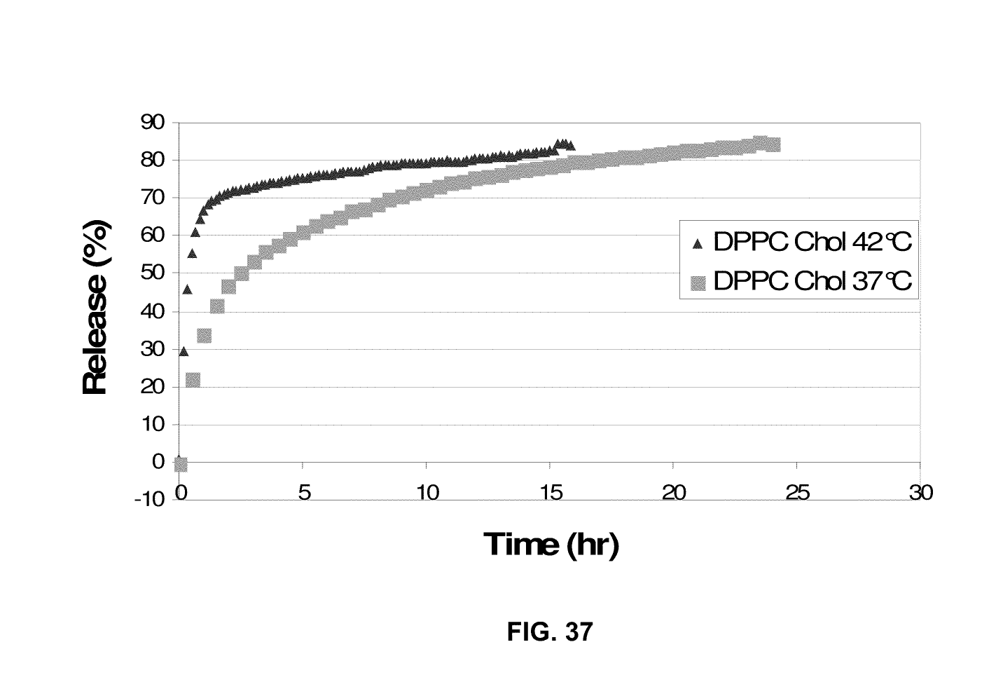

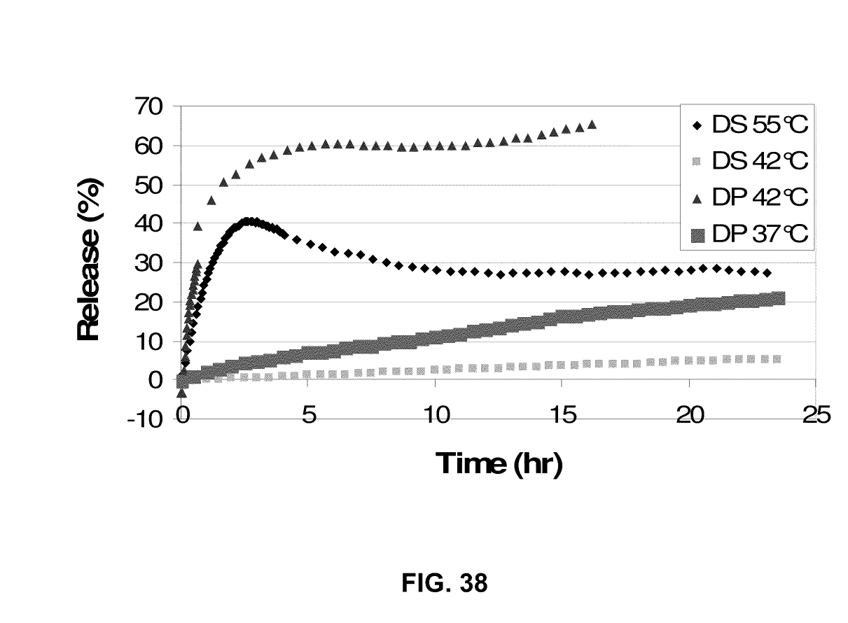

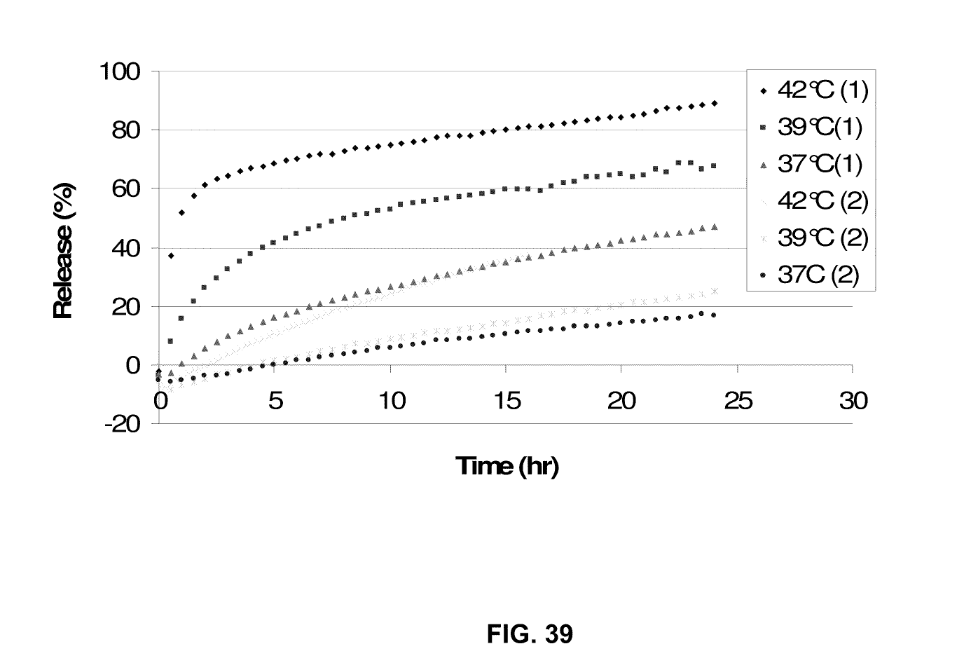

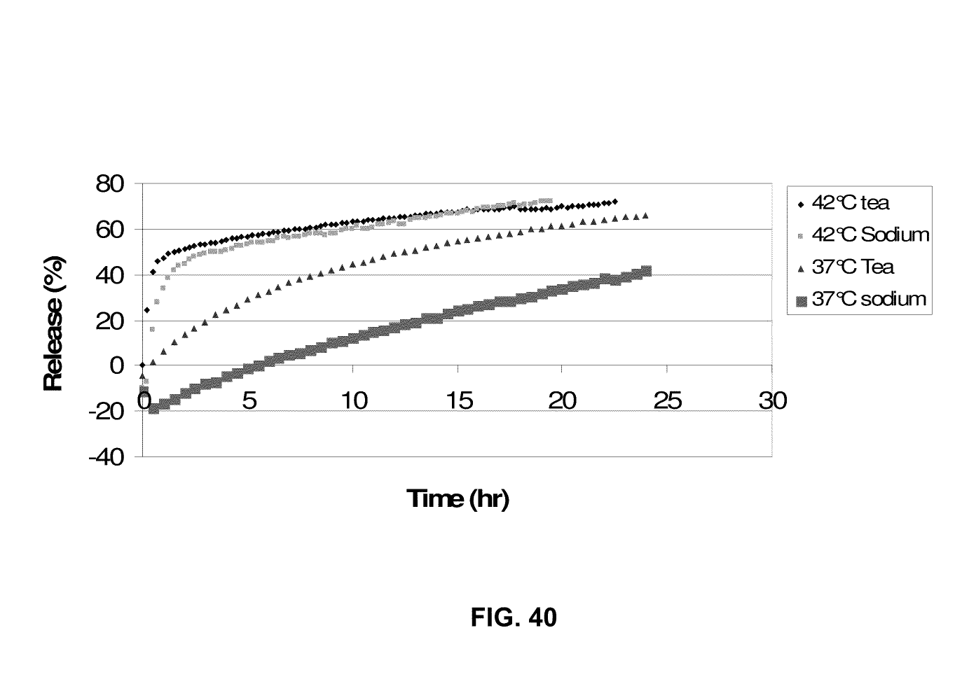

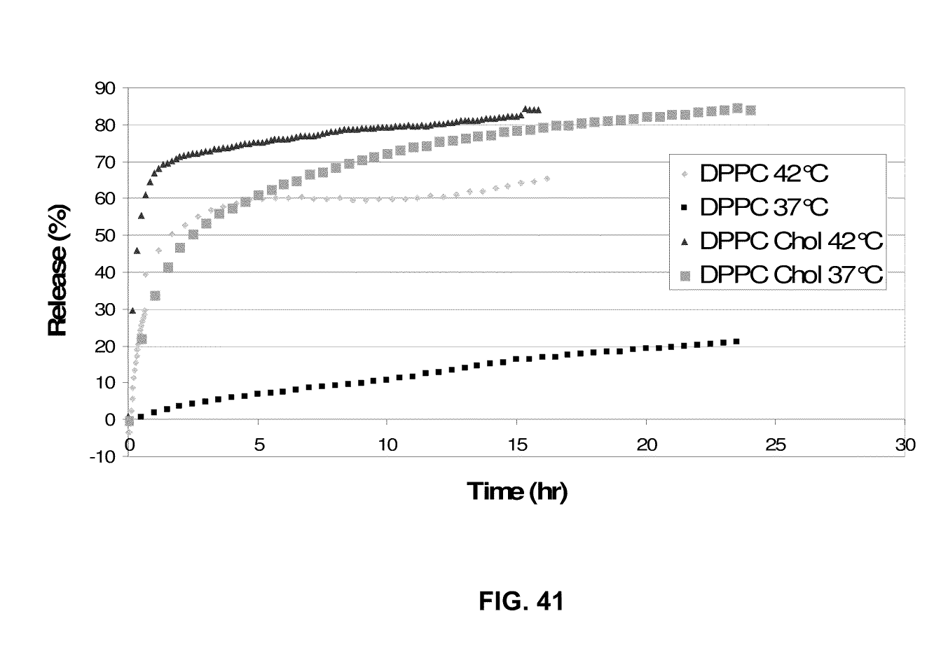

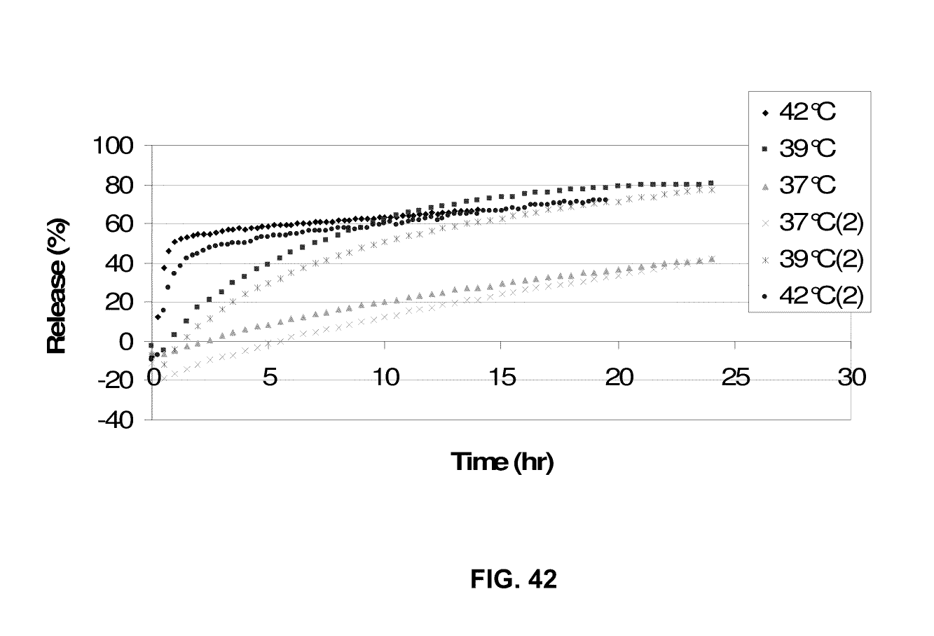

FIG. 24 shows the mechanism of flip-flop release from liposomes. At top, liposomes were formed with lipids containing a pyridyl disulfide functional group present on both outside and inside layers. TCEP selectively reduces the disulfide functional group on the outside layer while preserving the disulfides on the inside of the liposome. Bottom left—At the melting temperature of DPPC, the lipids undergo a flip-flop and switch layers. When the lipids are present in the same layer, a thiol-disulfide reaction occurs and releases pyridine-2-thione. FIG. 25 shows the chemical structure of 16:0 PDP-PE. FIG. 26 shows the chemical structure of 18:0 PDP-PE. FIG. 27 shows the chemical structure of DTP-2IT-DPPE. The release of chromophore by the flip-flop mediated mechanism of thiol-disulfide exchange is shown in FIGS. 28-42 and Tables 1-9 (below). For ease of presenting data to the reader, the lipid compositions of the liposomes are presented in the Tables and referenced in the figure legends. Experimental methodology is described in experimental section above.

### REFERENCES FOR EXAMPLE 6

(1) Matsumura, Y., and Maeda, H. (1986) A New Concept for Macromolecular Therapeutics in Cancer Chemotherapy: Mechanism of Tumoritropic Accumulation of Proteins and the Antitumor Agent Smancs. Cancer Res 46, 6387-6392.
(2) Milton, B. Y., Weinstein, J. N., Dennis, W. H., and Blumenthal, R. (1978) Design of Liposomes for Enhanced Local Release of Drugs by Hyperthermia. Science 202, 1290-1293.
(3) Saito, G., Swanson, J. A., and Lee, K.-D. (2003) Drug delivery strategy utilizing conjugation via reversible disulfide linkages: role and site of cellular reducing activities. Advanced Drug Delivery Reviews 55, 199-215.
(4) West, K. R., and Otto, S. (2005) in Current Drug Discovery Technologies pp 123-160, Bentham Science Publishers Ltd.
(5) Gurtovenko, A. A., and Vattulainen, I. (2007) Molecular Mechanism for Lipid Flip-Flops. The Journal of Physical Chemistry B 111, 13554-13559.
(6) John, K., Schreiber, S., Kubelt, J., Herrmann, A., and Müller, P. (2002) Transbilayer Movement of Phospholipids at the Main Phase Transition of Lipid Membranes: Implications for Rapid Flip-Flop in Biological Membranes. Biophysical Journal 83, 3315-3323.
(7) Regev, R., Yeheskely-Hayon, D., Katzir, H., and Eytan, G. D. (2005) Transport of anthracyclines and mitoxantrone across membranes by a flip-flop mechanism. Biochemical Pharmacology 70, 161-169.
(8) Brocklehurst, K., Kierstan, M., and Little, G. (1972) The reaction of papain with Ellman's reagent (5,5′-dithiobis-(2-nitrobenzoate) dianion). Biochem. J. 128, 811-816.
(9) Cline, D. J., Redding, S. E., Brohawn, S. G., Psathas, J. N., Schneider, J. P., and Thorpe, C. (2004) New Water-Soluble Phosphines as Reductants of Peptide and Protein Disulfide Bonds:&nbsp; Reactivity and Membrane Permeability&#x2020. Biochemistry 43, 15195-15203.
(10) Fiske, C. H., and Subbarow, Y. (1925) pp 375-400.
(11) Bartlett, G. R. (1959) pp 466-468.
(12) Udenfriend, S., Stein, S., BÃ¶hlen, P., Dairman, W., Leimgruber, W., and Weigele, M. (1972) pp 871-872, American Association for the Advancement of Science.
(13) Ellman, G. L. (1959) Tissue sulfhydryl groups. Archives of Biochemistry and Biophysics 82, 70-77.
(14) Riddles, P. W., Blakeley, R. L., Zemer, B., and Timasheff, C. H. W. H. a. S. N. (1983) [8] Reassessment of Ellman's reagent, in Methods in Enzymology pp 49-60, Academic Press.
(15) Brocklehurst, K., and Little, G. (1973) Reactions of papain and of low-molecular-weight thiols with some aromatic disulphides. 2,2′-Dipyridyl disulphide as a convenient active-site titrant for papain even in the presence of other thiols. Biochem. J. 133, 67-80.
(16) C. Nick Pace, F. V., Lanette Fee, Gerald Grimsley, Theronica Gray. (1995) How to measure and predict the molar absorption coefficient of a protein. Protein Science 4, 2411-2423.

TABLE 1
Lipid Composition

| Lipid | Mole % | μmol |
|---|---|---|
| DPPC | 88.4 | 15.3 |
| DSPE-PEG2000 | 5.8 | 1.0 |
| 16:0 PDP-PE | 5.8 | 1.0 |

TABLE 2
Lipid Composition

| Lipid | Mole % | μmol | mg of lipid | fw |
|---|---|---|---|---|
| DPPC | 88.4 | 15.3 | 12.77 | 734 |
| DSPE-PEG2000 | 5.8 | 1.0 | 2.86 | 2805.54 |
| DTP-2IT-DPPE | 5.8 | 1.0 | 0.90 | 902.2 |

TABLE 3
Lipid Composition

| Lipid | Mole % | μmol | mg of lipid | fw |
|---|---|---|---|---|
| DSPC | 88 | 15.3 | 11.2 | 734 |
| DSPE-PEG2000 | 6 | 1.0 | 2.86 | 2805.54 |
| 16:0 PDP-PE | 6 | 1.0 | 0.91 | 911.22 |

TABLE 4
Lipid Composition of Liposomes

| Lipid | Mole % | μmol |
|---|---|---|
| DPPC | 88 | 14.7 |
| DSPE-PEG2000 | 6 | 1.0 |
| 16:0 PDP-PE | 6 | 1.0 |

TABLE 5
Lipid Composition of Liposomes

| Lipid | Mole % | μmol |
|---|---|---|
| DPPC | 88 | 14.7 |
| DSPE-PEG2000 | 6 | 1.0 |
| 18:0 PDP-PE | 6 | 1.0 |

TABLE 6
Lipid Composition of Liposomes

| Lipid | Mole % | μmol |
|---|---|---|
| DPPC | 44 | 7.3 |
| DSPC | 44 | 7.3 |
| DSPE-PEG2000 | 6 | 1.0 |
| 16:0 PDP-PE | 6 | 1.0 |

TABLE 7
Lipid Composition of Liposomes

| Lipid | Mole % | μmol |
|---|---|---|
| DPPC | 44 | 7.3 |
| DSPC | 44 | 7.3 |
| DSPE-PEG2000 | 6 | 1.0 |
| 18:0 PDP-PE | 6 | 1.0 |

TABLE 8
Lipid Composition of Liposomes

| Lipid | Mole % | μmol |
|---|---|---|
| DPPC | 82 | 6.8 |
| DSPE-PEG2000 | 6 | 0.5 |
| 18:0 PDP-PE | 12 | 1.0 |

TABLE 9
Lipid Composition of Liposomes

| Lipid | Mole % | μmol |
|---|---|---|
| DPPC | 68 | 11.33 |
| DSPE-PEG2000 | 6 | 1.0 |
| 16:0 PDP-PE | 6 | 1.0 |
| Cholesterol | 20 | 3.33 |

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A composition, comprising:
a vehicle comprising one or more layers entrapping a liquid, a solid, or a combination thereof, wherein the one or more layers comprise an interior surface and an exterior surface, and wherein the interior surface contacts the entrapped liquid, solid, or combination thereof;
an agent coupled to the exterior surface via a cleavable linker; and
a cleaving molecule coupled to the exterior surface, wherein the cleavable linker and the cleaving molecule are present in a plurality of distinct domains on the exterior surface at a first temperature, the plurality of distinct domains being capable of mixing upon heating of the composition to a second temperature greater than the first temperature.

2. The composition of claim 1, wherein the second temperature is greater than or equal to a phase transition temperature.

3. The composition of claim 1, wherein the heating of the composition to the second temperature allows for cleavage of the cleavable linker by the cleaving molecule.

4. The composition of claim 1, wherein the one or more layers comprises a phospholipid.

5. The composition of claim 4, wherein the phospholipid is selected from the group consisting of: dimyristoylphosphatidyl choline, palmitoylmyristoylphosphatidyl choline, myristolypalmitoylphosphatidyl choline, dipalmitoylphosphatidyl choline, stearoylpalmitoylphosphatidyl choline, palmitoylstearolyphosphatidyl choline, distearolyphosphatidyl choline, and synthetic C₁₇ phosphatidyl choline.

6. The composition of claim 1, wherein the cleavable linker comprises a substrate.

7. (canceled)

8. The composition of claim 1, wherein the cleaving molecule comprises an enzyme.

9. (canceled)

10. The composition of claim 1, wherein the cleaving molecule comprises a thiol.

11. The composition of claim 1, wherein the second temperature comprises a range of from 38° C. to 80° C.

12. (canceled)

13. (canceled)

14. The composition of claim 1, wherein the agent is a therapeutic agent.

15. (canceled)

16. The composition of claim 2, wherein the composition comprises a plurality of distinct vehicles, wherein each distinct vehicle comprises a distinct agent, and wherein each distinct vehicle comprises a distinct phase transition temperature.

17. The composition of claim 1, wherein the heating is directed to a location at which release of the agent is desired.

18. The composition of claim 1, further comprising an indicator, wherein the indicator is for monitoring a thermal treatment efficacy in a tissue, quantifying a release of an agent, quantifying a thermal dose, quantifying a blood flow in a thermally-treated region, quantifying a systemic vehicle concentration, quantifying a systemic concentration of a released agent, or quantifying a ratio of released to intact vehicles.

19. The composition of claim 1, wherein the vehicle is a liposome.

20. The composition of claim 1, wherein the vehicle is a micelle.

21. (canceled)

22. The composition of claim 1, wherein the one or more layers comprises a lipid bilayer comprising an inner shell and an outer shell, wherein the inner shell comprises the interior surface and the outer shell comprises the exterior surface.

23. The composition of claim 1, wherein the vehicle does not comprise a fatty-acyl peptide, wherein the vehicle is not a microbubble, wherein the vehicle does not comprise a gas, and wherein pressure does not cause mixing of the plurality of distinct domains.

24. An ultrasound heating device, comprising:
a temperature feedback device that senses a temperature and provides a temperature-dependent signal interpretable by an ultrasound imaging device;
an acoustic pressure feedback device that senses acoustic pressure and provides an acoustic pressure-dependent signal interpretable by the ultrasound imaging device; and
a housing for the temperature and acoustic pressure feedback devices.

25. (canceled)

26. (canceled)

27. (canceled)

28. A method for treating a subject, comprising:
administering the composition of claim 1 to the subject;
allowing the composition to accumulate in a target site of the subject for a time period;
heating the target site to the second temperature with a heating device; and
releasing the agent from the composition, wherein the agent is released by the cleavage of the cleavable linker by the cleaving molecule.

29. (canceled)

30. A composition, comprising:
a vehicle comprising one or more layers entrapping a liquid, a solid, or a combination thereof, wherein the one or more layers comprise an interior surface and an exterior surface, and wherein the interior surface contacts the entrapped liquid, solid, or combination thereof;
an agent coupled to the exterior surface via a cleavable linker; and
a cleaving molecule coupled to the interior surface, wherein the cleavable linker is substantially present on the exterior surface at a first temperature, wherein the cleaving molecule is substantially present on the interior surface at the first temperature, and wherein the surfaces are capable of mixing upon heating of the composition to a second temperature greater than the first temperature.

31.-50. (canceled)
